(19) United States
(12) Patent Application Publication
(10) Pub. No.: US 2014/0227219 A1
(43) Pub. Date: Aug. 14, 2014

(54) Compositions and Methods Comprising BMP-7

(71) Applicant: University of Central Florida Research Foundation, Inc., Orlando, FL (US)

(72) Inventor: Dinender Singla, Orlando, FL (US)

(73) Assignee: University of Central Florida Research Foundation, Inc., Orlando, FL (US)

(21) Appl. No.: 14/176,085

(22) Filed: Feb. 8, 2014

Related U.S. Application Data
(60) Provisional application No. 61/762,642, filed on Feb. 8, 2013.

(51) Int. Cl. A61K 38/18; A61K 38/19; A61K 45/06

(52) U.S. Cl. CPC A61K 38/1875; A61K 45/06; A61K 38/193
USPC 424/85.1; 514/8.8; 514/1.9

(57) ABSTRACT

In an aspect, the invention relates to compositions comprising BMP-7 and methods of using disclosed compositions. In an aspect, the invention relates to compositions for and methods of treating atherosclerosis. This abstract is intended as a scanning tool for purposes of searching in the particular art and is not intended to be limiting of the present invention.

### CROSS-REFERENCE TO RELATED APPLICATIONS

This application claims the benefit of priority of U.S. Provisional Patent Application No. 61/762,642 filed Feb. 8, 2013, which is incorporated herein by reference it its entirety.

### BACKGROUND OF THE INVENTION

Atherosclerosis is a progressive disease, in which lesions of the arteries are formed by accumulation of plaque and neointimal hyperplasia causing an obstruction of blood flow. Often plaque is friable and may naturally dislodge, leading to an embolization of a downstream vessel. Accumulating evidence indicates that atherosclerosis is a chronic inflammatory disease. The innate immune cells that are involved in the pathogenesis of atherosclerosis are circulating monocytes and plaque macrophages. Complex interplay between immune and metabolic processes results in pathological activity of these cells.

Despite advances in the understanding of the pathology of atherosclerosis, there is still a need for compositions and methods that efficiently treat or prevent disease progression.

### BRIEF SUMMARY OF THE INVENTION

Disclosed herein are compositions for modulating polarization of a population of monocytes, comprising an effective amount of BMP-7, or a second agent that modulates the polarization of monocytes, or M-CSF, or a combination thereof.

Disclosed herein are compositions for ameliorating disease progression in a subject diagnosed with atherosclerosis, comprising an effective amount of BMP-7, or a second agent that modulates the polarization of monocytes, or M-CSF, or a combination thereof.

Disclosed herein are methods of ameliorating disease progression in a subject diagnosed with atherosclerosis, comprising administering to a subject a composition comprising an effective amount of BMP-7; thereby modulating the polarization of infiltrating monocytes in a subject.

Disclosed herein are methods of modulating polarization of a population of monocytes in a subject, comprising administering a composition comprising an effective amount of BMP-7 to a population of monocytes, thereby modulating the polarization of the monocytes into M1 macrophages and M2 macrophages.

Disclosed herein are methods of modulating BMP receptor expression in monocytes, comprising administering a composition comprising an effective amount of BMP-7 to a population of monocytes; and increasing the expression of a BMP receptor in the monocytes.

Disclosed herein are methods of modulating polarization of a population of monocytes, comprising administering a composition comprising an effective amount of BMP-7 to a population of monocytes; and modulating the polarization of the monocytes into M1 macrophages and M2 macrophages.

Disclosed herein are methods of modulating cellular phenotype, comprising administering a composition comprising an effective amount of BMP-7 to a population of macrophages; and modulating the cellular phenotype of the macrophages such that the macrophages exhibit characteristics of M2 macrophages.

Disclosed herein are methods of ameliorating disease progression in a subject in need thereof, comprising administering a composition comprising an effective amount of BMP-7 to a subject; altering the differentiation of infiltrating monocytes in a subject; and increasing the expression of anti-inflammatory cytokines in a subject.

Disclosed herein are methods of enhancing cardiac tissue regeneration or cell regeneration in a subject in need thereof, comprising administering a composition comprising an effective amount of BMP-7 to a subject; altering the differentiation of infiltrating monocytes in a subject; and increasing the expression of anti-inflammatory cytokines in a subject.

Disclosed herein are methods of attenuating atherosclerosis in a subject, comprising administering a composition comprising an effective amount of BMP-7 to a subject; altering the differentiation of infiltrating monocytes in a subject; and modulating the expression or activity of one or more anti-inflammatory cytokines in a subject.

### BRIEF DESCRIPTION OF THE SEVERAL VIEWS OF THE DRAWINGS

The accompanying figures, which are incorporated in and constitute a part of this specification, illustrate several aspects and together with the description serve to explain the principles of the invention.

FIG. 1 shows that BMP-7 treatment of THP-1 cells increased IL-10 activity.

FIG. 2 shows that BMP-7 treatment of THP-1 cells increased the % of BMP-7Rs on cells 48 hr after treatment.

FIG. 3A-FIG. 3D show that BMP-7 treatment reduced atherosclerotic plaque formation.

FIG. 4A-FIG. 4B quantify CD14 immunostaining for monocytes in and around the artery following PLCA.

FIG. 5A-FIG. 5B show that BMP-7 treatment had no effect of M1 macrophage differentiation following PLCA.

FIG. 6A-FIG. 6B show that BMP-7 treatment induced M2 macrophage differentiation following PLCA.

FIG. 7 shows that BMP-7 treatment increased expression of BMPR2 on monocytes following PLCA.

FIG. 8 shows that BMP-7 treatment increased expression of BMPR2 on M2 macrophages following PLCA.

FIG. 9A-FIG. 9B show that BMP-7 treatment increased levels of anti-inflammatory cytokines IL-10 and IL-1ra in blood plasma following PLCA.

FIG. 10 shows BMP-7 levels in blood following PLCA with and without BMP-7 treatment.

FIG. 11 shows that BMP-7 treatment increased SMAD activity following PLCA.

FIG. 12 shows that BMP-7 treatment improved left common carotid artery velocity function following PLCA.

FIG. 13A-FIG. 13D show the effect of BMP-7 treatment on BMPR2 expression in monocytes and macrophages.

FIG. 14A-FIG. 14E show the effect of BMP-7 treatment on anti-inflammatory and pro-inflammatory cytokine activity levels.

FIG. 15 shows that BMP-7 treatment increased expression of BMPR2 on monocytes.

FIG. 16 shows that BMP-7 treatment increased expression of BMPR2 on M2 macrophages.

FIG. 17A-FIG. 17D show that BMP-7 treatment decreased expression of the M1 macrophage marker iNOS and decreased expression of pro-inflammatory cytokines.

FIG. 18A-FIG. 18C show that BMP-7 treatment increased polarization of monocytes into M2 macrophages and increased expression of anti-inflammatory cytokines.

FIG. 19A-FIG. 19B show that LY-294002 treatment decreased expression of anti-inflammatory cytokines.

FIG. 20A-FIG. 20B show that binding of BMP-7 to BMPR2 phosphorylated SMAD-1, SMAD-5, SMAD-8 and activated PI3K.

FIG. 21A-FIG. 21C show that activation of PI3K resulted in phosphorylation of Akt and mTOR inhibition via PTEN.

FIG. 22 shows a schematic of a mechanism by which BMP-7 polarizes monocytes.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION OF THE INVENTION

The present invention comprises compositions comprising BMP-7, a second agent that modulates the polarization of monocytes, M-CSF, or a combination thereof and methods of using the disclosed compositions.

Though not wishing to be bound by any particular theory, it is thought that the development, progression, and pathogenesis of atherosclerosis (ATH) is complex and multifactorial and involves abnormal lipid metabolism, endothelial dysfunction, macrophage foam cell formation, and inflammation (Kleeman et al., 2008; Moore et al., 2011; Wolfs et al., 2011; Maiseyeu et al. 2010; Parthasarathy et al., 1988; Parthasarathy et al., 1994). ATH is a non-resolving inflammatory process as monocytes continue to enter in coronary artery at all stages of the disease. Inflammation is associated with infiltration of monocytes into the sub-endothelial space of large arteries and their differentiation into macrophages (Kleeman et al., 2008; Moore et al., 2011; Wolfs et al., 2011; Maiseyeu et al. 2010; Parthasarathy et al., 1988; Parthasarathy et al., 1994). The stimuli received from the microenvironment distinguish the classification of infiltrating monocytes (Moore et al., 2011; Wolfs et al., 2011). In ATH, monocytes differentiate into pro-inflammatory (M1) macrophages and anti-inflammatory (M2) macrophages (Kleeman et al., 2008; Moore et al., 2011; Wolfs et al., 2011). The pro-inflammatory M1 macrophages are known to induce tissue damage and impair wound healing (Kigerl et al., 2009). Macrophages respond to the various stimuli received from their own microenvironment or via treatment, which results in an enhanced inflammatory immune response and cell plasticity (Kleeman et al., 2008; Wolfs, et al., 2011; Kadl et al., 2010; Lovren et al., 2010; Yamamoto et al., 2011). Therefore, the differentiation of monocytes into the anti-inflammatory M2 macrophages can promote tissue repair and healing.

Though not wishing to be bound by any particular theory, it is thought that bone morphogenic proteins or BMPs belong to the family of transforming growth factor-β (TGF-β), which has 20 members (Bragdon et al., 2011). BMP-7 is a key growth factor responsible for directing differentiation of mesenchymal stem cells (MSCs) into brown fat cells (Tseng et al., 2008). BMP-7 may be involved in the development and maintenance of various physiological processes and cell differentiation in the body (Boon et al., 2001).

Compositions

Disclosed herein are compositions comprising BMP-7, a second agent that modulates the polarization of monocytes, M-CSF, or a combination thereof. In an aspect, a disclosed composition can comprise at least one second agent that modulates the polarization of monocytes, including but not limited, to M-CSF. In an aspect, a disclosed composition can comprise more than one second agent that modulates the polarization of monocytes. For example, in an aspect, a disclosed composition can comprise BMP-7 and at least one second agent that modulates the polarization of monocytes. In an aspect, a disclosed composition can comprise BMP-7 and M-CSF. In an aspect, a disclosed composition can comprise BMP-7, at least one second agent that modulates the polarization of monocytes, and M-CSF. In an aspect, a disclosed composition does not comprise M-CSF. In an aspect, compositions disclosed herein can comprise an amount of BMP-7 effective for obtaining a desired result. In an aspect, compositions disclosed herein can comprise at least one pharmaceutically acceptable carrier. In an aspect, a disclosed composition can be a pharmaceutical composition.

A second agent that modulates the polarization of monocytes can be a compound, a protein, a nucleic acid, a factor, a biological substance, or a chemical substance, or a complex thereof. In an aspect, a second agent that modulates the polarization of monocytes can be M-CSF. In an aspect, a second agent that modulates the polarization of monocytes is not M-CSF. In an aspect, a second agent that modulates the polarization of monocytes can polarize monocytes independently of BMP-7.

In an aspect, a disclosed composition can be formulated to be administered by any route known to the art. In an aspect, a disclosed composition can be formulated for intravenous administration. In an aspect, a disclosed composition can be formulated for administration via a route other than intravenous such as, for example, oral, local, transdermal, intradermal, intraperitoneal, intrathecal, intraocular, nasal (i.e., via inhalation), topical, intravaginal, ophthalmical, intraaural, intracerebral, rectal, sublingual, parenteral, and via the ears and mouth. In an aspect, a disclosed composition can be formulated to be administered by one or more routes of administration known to the art.

In an aspect, a disclosed composition can be formulated to be administered one or more times, in a rapid or sustained delivery formulation. In an aspect, administration of a disclosed composition can be repeated hourly, daily, weekly, bi-weekly, monthly, bi-monthly, annually, every other year, or at any other interval deemed appropriate by the skilled person in the art. In an aspect, a composition can be continuously administered.

Methods

Disclosed herein is a method of modulating polarization of a population of monocytes, comprising administering a composition comprising an effective amount of BMP-7, thereby modulating the polarization of the monocytes. In an aspect, an effective amount of BMP-7 is effective for modulating the polarization of monocytes into M1 and M2 macrophages. In an aspect, modulating the polarization of monocytes can comprise increasing the rate of or the extent of polarization of the monocytes into M2 macrophages. In an aspect, modulating the polarization of monocytes can comprise decreasing the rate of or the extent of polarization of monocytes into M1 macrophages.

In an aspect, a disclosed method can further comprise administering at least one second agent that modulates the polarization of monocytes. In an aspect, a second agent can be M-CSF. In an aspect, a second agent is not M-CSF. In an aspect, when a second agent is not M-CSF, a disclosed method can further comprise administering M-CSF. For example, disclosed herein is a method comprising administering a composition comprising an effective amount of BMP-7, a second agent that modulates the polarization of monocytes that is not M-CSF, and M-CSF.

When administered with (i) at least one second agent that modulates the polarization of monocytes, or (ii) M-CSF, or (iii) a combination thereof, a disclosed composition comprising an effective amount of BMP-7 can be administered prior to, concurrent with, or subsequent to the administration of (i) at least one second agent, (ii) M-CSF, or (ii) a combination thereof.

As used herein, “a combination thereof” may mean (i) a composition comprising a combination of BMP-7 with at least one second agent that modulates the polarization of monocytes that is not M-CSF, (ii) a composition comprising a combination of BMP-7 with M-CSF, (iii) a composition comprising a combination of BMP-7, at least one second agent that modulates the polarization of monocytes that is not M-CSF, and M-CSF, or (iv) a composition comprising a combination of a second agent that modulates the polarization of monocytes that is not M-CSF and M-CSF.

In an aspect, a disclosed method can be performed in vitro or in vivo. In an aspect, the monocytes are in a subject and a disclosed method is performed in vivo. In an aspect, a subject to be treated by a disclosed method can be diagnosed with atherosclerosis, or myocardial infarction, or myocarditis, or a cardiovascular disease, or condition that comprises an immune response, or a condition that comprises macrophage infiltration. In an aspect, an immune response can be anti-inflammatory, can be pro-inflammatory, or can be both anti-inflammatory and pro-inflammatory.

In an aspect, a subject can be diagnosed with one or more of the following: atherosclerosis, myocardial infarction, myocarditis, cardiovascular disease, a condition that comprises an immune response, and/or a condition that comprises macrophage infiltration.

In an aspect of a disclosed method, a composition can be administered to a subject by any route known to the art. In an aspect, a disclosed composition can be administered intravenously. In an aspect, administration can be via a route other than intravenously such as, for example, orally, locally, transdermally, intradermally, intraperitoneally, intrathecally, intraocularly, nasally (i.e., via inhalation), topically, intravaginally, ophthalmically, intraaurally, intracerebrally, rectally, sublingually, parenterally, and via the ears and mouth.

In an aspect of a disclosed method, a composition can be administered to a subject one or more times. In an aspect, administration can be repeated hourly, daily, weekly, bi-weekly, monthly, bi-monthly, annually, every other year, or at any other interval deemed appropriate by the skilled person in the art. In an aspect of a disclosed method, a composition can be administered continuously.

Disclosed herein is a method of modulating BMP receptor expression in monocytes, comprising administering a composition comprising an effective amount of BMP-7 to a population of monocytes; and increasing the expression of a BMP receptor in the monocytes. In an aspect, an effective amount of BMP-7 is effective for increasing the expression of a BMP receptor in the monocytes. For example, in an aspect, a BMP receptor can be a BMP Type II receptor (BMPR2). In an aspect, a BMP receptor can be a BMP Type I receptor (BMPR1).

In an aspect, a disclosed method can further comprise administering at least one second agent that modulates the polarization of monocytes. In an aspect, a second agent can be M-CSF. In an aspect, a second agent is not M-CSF. In an aspect, when a second agent is not M-CSF, a disclosed method can further comprise administering M-CSF. For example, disclosed herein is a method comprising administering a composition comprising an effective amount of BMP-7, at least a second agent that modulates the polarization of monocytes that is not M-CSF, and M-CSF.

When administered with (i) at least one second agent that modulates the polarization of monocytes, or (ii) M-CSF, or (iii) a combination thereof, a disclosed composition comprising an effective amount of BMP-7 can be administered prior to, concurrent with, or subsequent to the administration of (i) the at least one second agent, (ii) M-CSF, or (ii) a combination thereof.

As used herein, “a combination thereof” may mean (i) a composition comprising a combination of BMP-7 with at least one second agent that modulates the polarization of monocytes that is not M-CSF, (ii) a composition comprising a combination of BMP-7 with M-CSF, (iii) a composition comprising a combination of BMP-7, at least one second agent that modulates the polarization of monocytes that is not M-CSF, and M-CSF, or (iv) a composition comprising a combination of a second agent that modulates the polarization of monocytes that is not M-CSF and M-CSF.

In an aspect, a disclosed method can be performed in vitro or in vivo. In an aspect, the monocytes are in a subject and a disclosed method is performed in vivo. In an aspect, a subject to be treated by a disclosed method can be diagnosed with atherosclerosis, or myocardial infarction, or myocarditis, or a cardiovascular disease, or condition that comprises an immune response, or a condition that comprises macrophage infiltration. In an aspect, an immune response can be anti-inflammatory, can be pro-inflammatory, or can be both anti-inflammatory and pro-inflammatory.

In an aspect, a subject can be diagnosed with one or more of the following: atherosclerosis, myocardial infarction, myocarditis, cardiovascular disease, a condition that comprises an immune response, and/or a condition that comprises macrophage infiltration.

In an aspect of a disclosed method, a composition can be administered to a subject by any route known to the art. In an aspect, a disclosed composition can be administered intravenously. In an aspect, administration can be via a route other than intravenously such as, for example, orally, locally, transdermally, intradermally, intraperitoneally, intrathecally, intraocularly, nasally (i.e., via inhalation), topically, intravaginally, ophthalmically, intraaurally, intracerebrally, rectally, sublingually, parenterally, and via the ears and mouth.

In an aspect of a disclosed method, a composition can be administered to a subject one or more times. In an aspect, administration can be repeated hourly, daily, weekly, bi-weekly, monthly, bi-monthly, annually, every other year, or at any other interval deemed appropriate by the skilled person in the art. In an aspect of a disclosed method, a composition can be administered continuously.

Disclosed herein is a method of modulating cellular phenotype, comprising administering a composition comprising an effective amount of BMP-7 to a population of macrophages; and modulating the cellular phenotype of the macrophages such that the macrophages exhibit characteristics of M2 macrophages. In an aspect, an effective amount of BMP-7 is effective for modulating the cellular phenotype of the macrophages such that the macrophages exhibit characteristics of M2 macrophage.

In an aspect, a disclosed method can further comprise administering at least one second agent that modulates the polarization of monocytes. In an aspect, a second agent can be M-CSF. In an aspect, a second agent is not M-CSF. In an aspect, when a second agent is not M-CSF, a disclosed method can further comprise administering M-CSF. For example, disclosed herein is a method comprising administering a composition comprising an effective amount of BMP-7, at least a second agent that modulates the polarization of monocytes that is not M-CSF, and M-CSF.

When administered with (i) at least one second agent that modulates the polarization of monocytes, or (ii) M-CSF, or (iii) a combination thereof, a disclosed composition comprising an effective amount of BMP-7 can be administered prior to, concurrent with, or subsequent to the administration of (i) the at least one second agent, (ii) M-CSF, or (ii) a combination thereof.

As used herein, “a combination thereof” may mean (i) a composition comprising a combination of BMP-7 with at least one second agent that modulates the polarization of monocytes that is not M-CSF, (ii) a composition comprising a combination of BMP-7 with M-CSF, (iii) a composition comprising a combination of BMP-7, at least one second agent that modulates the polarization of monocytes that is not M-CSF, and M-CSF, or (iv) a composition comprising a combination of a second agent that modulates the polarization of monocytes that is not M-CSF and M-CSF.

In an aspect, a disclosed method can be performed in vitro or in vivo. In an aspect, the monocytes are in a subject and a disclosed method is performed in vivo. In an aspect, a subject to be treated by a disclosed method can be diagnosed with atherosclerosis, or myocardial infarction, or myocarditis, or a cardiovascular disease, or condition that comprises an immune response, or a condition that comprises macrophage infiltration. In an aspect, an immune response can be anti-inflammatory, can be pro-inflammatory, or can be both anti-inflammatory and pro-inflammatory.

In an aspect, a subject can be diagnosed with one or more of the following: atherosclerosis, myocardial infarction, myocarditis, cardiovascular disease, a condition that comprises an immune response, and/or a condition that comprises macrophage infiltration.

In an aspect of a disclosed method, a composition can be administered to a subject by any route known to the art. In an aspect, a disclosed composition can be administered intravenously. In an aspect, administration can be via a route other than intravenously such as, for example, orally, locally, transdermally, intradermally, intraperitoneally, intrathecally, intraocularly, nasally (i.e., via inhalation), topically, intravaginally, ophthalmically, intraaurally, intracerebrally, rectally, sublingually, parenterally, and via the ears and mouth.

In an aspect of a disclosed method, a composition can be administered to a subject one or more times. In an aspect, administration can be repeated hourly, daily, weekly, bi-weekly, monthly, bi-monthly, annually, every other year, or at any other interval deemed appropriate by the skilled person in the art. In an aspect of a disclosed method, a composition can be administered continuously.

Disclosed herein is a method of ameliorating disease progression in a subject diagnosed with atherosclerosis, comprising administering to a subject a composition comprising an effective amount of BMP-7; thereby modulating the polarization of infiltrating monocytes in a subject. In an aspect, an effective amount of BMP-7 is effective for modulating the polarization of infiltrating monocytes. In an aspect, modulating the polarization of monocytes can comprise increasing the rate of or extent of polarization of the monocytes into M2 macrophages. In an aspect, modulating the polarization of monocytes can comprise decreasing the rate of or extent of polarization of monocytes into M1 macrophages.

In an aspect, a disclosed method can further comprise administering at least one second agent that modulates the polarization of monocytes. In an aspect, a second agent can be M-CSF. In an aspect, a second agent is not M-CSF. In an aspect, when a second agent is not M-CSF, a disclosed method can further comprise administering M-CSF. For example, disclosed herein is a method comprising administering a composition comprising an effective amount of BMP-7, at least a second agent that modulates the polarization of monocytes that is not M-CSF, and M-CSF.

When administered with (i) at least one second agent that modulates the polarization of monocytes, or (ii) M-CSF, or (iii) a combination thereof, a disclosed composition comprising an effective amount of BMP-7 can be administered prior to, concurrent with, or subsequent to the administration of (i) at least one second agent, (ii) M-CSF, or (ii) a combination thereof.

As used herein, “a combination thereof” may mean (i) a composition comprising a combination of BMP-7 with at least one second agent that modulates the polarization of monocytes that is not M-CSF, (ii) a composition comprising a combination of BMP-7 with M-CSF, (iii) a composition comprising a combination of BMP-7, at least one second agent that modulates the polarization of monocytes that is not M-CSF, and M-CSF, or (iv) a composition comprising a combination of a second agent that modulates the polarization of monocytes that is not M-CSF and M-CSF.

In an aspect, a disclosed method can be performed in vitro or in vivo. In an aspect, the monocytes are in a subject and a disclosed method is performed in vivo. In an aspect, a subject to be treated by a disclosed method can be diagnosed with atherosclerosis, or myocardial infarction, or myocarditis, or a cardiovascular disease, or condition that comprises an immune response, or a condition that comprises macrophage infiltration. In an aspect, an immune response can be anti-inflammatory, can be pro-inflammatory, or can be both anti-inflammatory and pro-inflammatory.

In an aspect, a subject can be diagnosed with one or more of the following: atherosclerosis, myocardial infarction, myocarditis, cardiovascular disease, a condition that comprises an immune response, and/or a condition that comprises macrophage infiltration.

In an aspect of a disclosed method, a composition can be administered to a subject by any route known to the art. In an aspect, a disclosed composition can be administered intravenously. In an aspect, administration can be via a route other than intravenously such as, for example, orally, locally, transdermally, intradermally, intraperitoneally, intrathecally, intraocularly, nasally (i.e., via inhalation), topically, intravaginally, ophthalmically, intraaurally, intracerebrally, rectally, sublingually, parenterally, and via the ears and mouth.

In an aspect of a disclosed method, a composition can be administered to a subject one or more times. In an aspect, administration can be repeated hourly, daily, weekly, bi-weekly, monthly, bi-monthly, annually, every other year, or at any other interval deemed appropriate by the skilled person in the art. In an aspect of a disclosed method, a composition can be administered continuously.

Disclosed herein is a method of attenuating atherosclerosis in a subject, comprising administering a composition comprising an effective amount of BMP-7 to a subject; altering the differentiation of infiltrating monocytes in a subject; and modulating the expression or activity of one or more anti-inflammatory cytokines in a subject. In an aspect, an effective amount of BMP-7 is effective for altering the differentiation of infiltration monocytes and/or modulating the expression or activity of one or more anti-inflammatory cytokines. In an aspect, altering the differentiation of infiltrating monocytes can comprise increasing the rate of or extent of polarization of the monocytes into M2 macrophages. In an aspect, altering the differentiation of infiltrating monocytes can comprise decreasing the rate of or extent of polarization of monocytes into M1 macrophages.

In an aspect, a disclosed method can further comprise administering at least one second agent that modulates the polarization of monocytes. In an aspect, a second agent can be M-CSF. In an aspect, a second agent is not M-CSF. In an aspect, when a second agent is not M-CSF, a disclosed method can further comprise administering M-CSF. For example, disclosed herein is a method comprising administering a composition comprising an effective amount of BMP-7, at least a second agent that modulates the polarization of monocytes that is not M-CSF, and M-CSF.

When administered with (i) at least one second agent that modulates the polarization of monocytes, or (ii) M-CSF, or (iii) a combination thereof, a disclosed composition comprising an effective amount of BMP-7 can be administered prior to, concurrent with, or subsequent to the administration of (i) at least one second agent, (ii) M-CSF, or (ii) a combination thereof.

As used herein, “a combination thereof” may mean (i) a composition comprising a combination of BMP-7 with at least one second agent that modulates the polarization of monocytes that is not M-CSF, (ii) a composition comprising a combination of BMP-7 with M-CSF, (iii) a composition comprising a combination of BMP-7, at least one second agent that modulates the polarization of monocytes that is not M-CSF, and M-CSF, or (iv) a composition comprising a combination of a second agent that modulates the polarization of monocytes that is not M-CSF and M-CSF.

In an aspect, a disclosed method can be performed in vitro or in vivo. In an aspect, the monocytes are in a subject and a disclosed method is performed in vivo. In an aspect, a subject to be treated by a disclosed method can be diagnosed with atherosclerosis, or myocardial infarction, or myocarditis, or a cardiovascular disease, or condition that comprises an immune response, or a condition that comprises macrophage infiltration. In an aspect, an immune response can be anti-inflammatory, can be pro-inflammatory, or can be both anti-inflammatory and pro-inflammatory.

In an aspect, a subject can be diagnosed with one or more of the following: atherosclerosis, myocardial infarction, myocarditis, cardiovascular disease, a condition that comprises an immune response, and/or a condition that comprises macrophage infiltration.

In an aspect of a disclosed method, a composition can be administered to a subject by any route known to the art. In an aspect, a disclosed composition can be administered intravenously. In an aspect, administration can be via a route other than intravenously such as, for example, orally, locally, transdermally, intradermally, intraperitoneally, intrathecally, intraocularly, nasally (i.e., via inhalation), topically, intravaginally, ophthalmically, intraaurally, intracerebrally, rectally, sublingually, parenterally, and via the ears and mouth.

In an aspect of a disclosed method, a composition can be administered to a subject one or more times. In an aspect, administration can be repeated hourly, daily, weekly, bi-weekly, monthly, bi-monthly, annually, every other year, or at any other interval deemed appropriate by the skilled person in the art. In an aspect of a disclosed method, a composition can be administered continuously.

Disclosed herein is a method of ameliorating disease progression in a subject in need thereof, comprising administering a composition comprising an effective amount of BMP-7 to a subject, altering the differentiation of infiltrating monocytes in a subject; and increasing the expression of anti-inflammatory cytokines in a subject. In an aspect, an effective amount of BMP-7 is effective for altering the differentiation of infiltrating monocytes and/or increasing the expression of anti-inflammatory cytokines in a subject. In an aspect, altering the differentiation of infiltrating monocytes can comprise increasing the rate of or extent of polarization of the monocytes into M2 macrophages. In an aspect, altering the differentiation of infiltrating monocytes can comprise decreasing the rate of or extent of polarization of monocytes into M1 macrophages.

In an aspect, a disclosed method can further comprise administering at least one second agent that modulates the polarization of monocytes. In an aspect, a second agent can be M-CSF. In an aspect, a second agent is not M-CSF. In an aspect, when a second agent is not M-CSF, a disclosed method can further comprise administering M-CSF. For example, disclosed herein is a method comprising administering a composition comprising an effective amount of BMP-7, at least a second agent that modulates the polarization of monocytes that is not M-CSF, and M-CSF.

When administered with (i) at least one second agent that modulates the polarization of monocytes, or (ii) M-CSF, or (iii) a combination thereof, a disclosed composition comprising an effective amount of BMP-7 can be administered prior to, concurrent with, or subsequent to the administration of (i) at least one second agent, (ii) M-CSF, or (ii) a combination thereof.

As used herein, “a combination thereof” may mean (i) a composition comprising a combination of BMP-7 with at least one second agent that modulates the polarization of monocytes that is not M-CSF, (ii) a composition comprising a combination of BMP-7 with M-CSF, (iii) a composition comprising a combination of BMP-7, at least one second agent that modulates the polarization of monocytes that is not M-CSF, and M-CSF, or (iv) a composition comprising a combination of a second agent that modulates the polarization of monocytes that is not M-CSF and M-CSF.

In an aspect, a disclosed method can be performed in vitro or in vivo. In an aspect, the monocytes are in a subject and a disclosed method is performed in vivo. In an aspect, a subject to be treated by a disclosed method can be diagnosed with atherosclerosis, or myocardial infarction, or myocarditis, or a cardiovascular disease, or condition that comprises an immune response, or a condition that comprises macrophage infiltration. In an aspect, an immune response can be anti-inflammatory, can be pro-inflammatory, or can be both anti-inflammatory and pro-inflammatory.

In an aspect, a subject can be diagnosed with one or more of the following: atherosclerosis, myocardial infarction, myocarditis, cardiovascular disease, a condition that comprises an immune response, and/or a condition that comprises macrophage infiltration.

In an aspect of a disclosed method, a composition can be administered to a subject by any route known to the art. In an aspect, a disclosed composition can be administered intravenously. In an aspect, administration can be via a route other than intravenously such as, for example, orally, locally, transdermally, intradermally, intraperitoneally, intrathecally, intraocularly, nasally (i.e., via inhalation), topically, intravaginally, ophthalmically, intraaurally, intracerebrally, rectally, sublingually, parenterally, and via the ears and mouth.

In an aspect of a disclosed method, a composition can be administered to a subject one or more times. In an aspect, administration can be repeated hourly, daily, weekly, bi-weekly, monthly, bi-monthly, annually, every other year, or at any other interval deemed appropriate by the skilled person in the art. In an aspect of a disclosed method, a composition can be administered continuously.

Disclosed herein is a method of enhancing cardiac tissue regeneration or cell regeneration in a subject in need thereof, comprising administering a composition comprising an effective amount of BMP-7 to a subject; altering the differentiation of infiltrating monocytes in a subject; and increasing the expression of anti-inflammatory cytokines in a subject. In an aspect, an effective amount of BMP-7 is effective for altering the differentiation of infiltrating monocytes in a subject and/or increasing the expression of anti-inflammatory cytokines in a subject. In an aspect, altering the differentiation of infiltrating monocytes can comprise increasing the rate of or extent of polarization of the monocytes into M2 macrophages. In an aspect, altering the differentiation of infiltrating monocytes can comprise decreasing the rate of or extent of polarization of monocytes into M1 macrophages.

In an aspect, a disclosed method can further comprise administering at least one second agent that modulates the polarization of monocytes. In an aspect, a second agent can be M-CSF. In an aspect, a second agent is not M-CSF. In an aspect, when a second agent is not M-CSF, a disclosed method can further comprise administering M-CSF. For example, disclosed herein is a method comprising administering a composition comprising an effective amount of BMP-7, at least a second agent that modulates the polarization of monocytes that is not M-CSF, and M-CSF.

When administered with (i) at least one second agent that modulates the polarization of monocytes, or (ii) M-CSF, or (iii) a combination thereof, a disclosed composition comprising an effective amount of BMP-7 can be administered prior to, concurrent with, or subsequent to the administration of (i) at least one second agent, (ii) M-CSF, or (ii) a combination thereof.

As used herein, “a combination thereof” may mean (i) a composition comprising a combination of BMP-7 with at least one second agent that modulates the polarization of monocytes that is not M-CSF, (ii) a composition comprising a combination of BMP-7 with M-CSF, (iii) a composition comprising a combination of BMP-7, at least one second agent that modulates the polarization of monocytes that is not M-CSF, and M-CSF, or (iv) a composition comprising a combination of a second agent that modulates the polarization of monocytes that is not M-CSF and M-CSF.

In an aspect, a disclosed method can be performed in vitro or in vivo. In an aspect, the monocytes are in a subject and a disclosed method is performed in vivo. In an aspect, a subject to be treated by a disclosed method can be diagnosed with atherosclerosis, or myocardial infarction, or myocarditis, or a cardiovascular disease, or condition that comprises an immune response, or a condition that comprises macrophage infiltration. In an aspect, an immune response can be anti-inflammatory, can be pro-inflammatory, or can be both anti-inflammatory and pro-inflammatory.

In an aspect, a subject can be diagnosed with one or more of the following: atherosclerosis, myocardial infarction, myocarditis, cardiovascular disease, a condition that comprises an immune response, and/or a condition that comprises macrophage infiltration.

In an aspect of a disclosed method, a composition can be administered to a subject by any route known to the art. In an aspect, a disclosed composition can be administered intravenously. In an aspect, administration can be via a route other than intravenously such as, for example, orally, locally, transdermally, intradermally, intraperitoneally, intrathecally, intraocularly, nasally (i.e., via inhalation), topically, intravaginally, ophthalmically, intraaurally, intracerebrally, rectally, sublingually, parenterally, and via the ears and mouth.

In an aspect of a disclosed method, a composition can be administered to a subject one or more times. In an aspect, administration can be repeated hourly, daily, weekly, bi-weekly, monthly, bi-monthly, annually, every other year, or at any other interval deemed appropriate by the skilled person in the art. In an aspect of a disclosed method, a composition can be administered continuously.

In an aspect of a disclosed method, modulating the polarization of monocytes can comprise increasing the rate of or extent of polarization of the monocytes into M2 macrophages. In an aspect, modulating the polarization of monocytes can comprise decreasing the rate of or extent of polarization of monocytes into M1 macrophages. In an aspect, as monocytes polarize into M2 macrophages, monocytes can phosphorylate proteins. In an aspect, phosphorylated proteins can comprise phosphorylated SMAD proteins. SMAD proteins can include, but are not limited to, SMAD-1, SMAD-5, SMAD-8, or a combination thereof. In an aspect, phosphorylation of SMAD proteins can activate related MAP Kinase proteins.

In an aspect of a disclosed method, increasing the expression of anti-inflammatory cytokines can comprise increasing the expression of one or more, two or more, three or more, four or more, five or more, of all of the following: IL-1ra, IL-4, IL-6, IL-10, IL-11, and IL-13.

Disease amelioration can be determined by a change in physiology after administration of a disclosed composition. For example, in an aspect, following the administration of a disclosed composition, severity of atherosclerosis can be reduced. In an aspect, following the administration of a disclosed composition, blood velocity can be improved. In an aspect, following the administration of a disclosed composition, formation of plaque can be reduced. In an aspect, following the administration of a disclosed composition, severity of atherosclerosis can be reduced, blood velocity can be improved, and formation of plaque can be reduced. In an aspect, following the administration of a disclosed composition, a combination of changes in physiology can be effected, such as, for example, severity of atherosclerosis can be reduced and blood velocity can be improved, or severity of atherosclerosis can be reduced and formation of plaque can be reduced, or blood velocity can be improved and formation of plaque can be reduced.

In an aspect of a disclosed method, following the administration of a disclosed composition, cardiac function can be enhanced. In an aspect, enhanced cardiac function can comprise improving left ventricular function, improving fractional shortening, improving ejection fraction, reducing end-diastolic volume, decreasing left ventricular mass, and normalizing of heart geometry. In an aspect, enhanced cardiac function can comprise a combination of improving left ventricular function, improving fractional shortening, improving ejection fraction, reducing end-diastolic volume, decreasing left ventricular mass, and normalizing of heart geometry.

For example, in an aspect, enhanced cardiac function can comprise one or more, two or more, three or more, four or more, five or more, or all of the following: improving left ventricular function, improving fractional shortening, improving ejection fraction, reducing end-diastolic volume, decreasing left ventricular mass, and normalizing of heart geometry.

### DEFINITIONS

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the amino acid abbreviations are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

“Peptide” as used herein refers to any peptide, oligopeptide, polypeptide, gene product, expression product, or protein. For example, a peptide can be an enzyme. A peptide is comprised of consecutive amino acids. Polypeptides encompass naturally occurring or synthetic molecule, and may contain modified amino acids. Polypeptides can be modified by either natural processes, such as post-translational processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in a given polypeptide.

In general, the biological activity or biological action of a gene/nucleic acid or peptide refers to any function exhibited or performed by the gene/nucleic acid or peptide that is ascribed to the naturally occurring form of the gene/nucleic acid or peptide as measured or observed in vivo (i.e., in the natural physiological environment of the gene/nucleic acid or peptide) or in vitro (i.e., under laboratory conditions).

The term “enzyme” as used herein refers to any peptide that catalyzes a chemical reaction of other substances without itself being destroyed or altered upon completion of the reaction. Typically, a peptide having enzymatic activity catalyzes the formation of one or more products from one or more substrates. Such peptides can have any type of enzymatic activity including, without limitation, the enzymatic activity or enzymatic activities associated with enzymes such as those disclosed herein.

Cells can be obtained from commercial sources such as the American Type Culture Collection (ATCC) and can be prokaryotic or eukaryotic. Cells can be grown in liquid media culture or on tissue culture plates. The growth conditions are dependent upon the specific cells used and such conditions would be known to one of skill in the art. Transfection and growth of cells are described in Maniatis et al.

As used herein, the terms “optional” or “optionally” means that the subsequently described event or circumstance can or cannot occur, and that the description includes instances where said event or circumstance occurs and instances where it does not. For example, in an aspect, a disclosed composition comprising BMP-7 may optionally comprise at least one second agent that modulates the polarization of monocytes. In an aspect, the at least one second agent can optionally be M-CSF.

The term “contacting” as used herein refers to bringing a disclosed compound and a cell, target receptor, gene, peptide, or other biological entity together in such a manner that the compound can affect the activity of the target (e.g., receptor, transcription factor, cell, etc.), either directly by interacting with the target itself, or indirectly by interacting with another molecule, co-factor, factor, or protein on which the activity of the target is dependent.

As used herein, the term “determining” can refer to measuring or ascertaining a quantity or an amount or a change in expression and/or in activity level or in prevalence. For example, determining can refer to measuring or ascertaining the quantity or amount of a nucleotide or transcript or polypeptide or pro-inflammatory cytokine or anti-inflammatory cytokine. For example, determining can refer to measuring or ascertaining the prevalence of a particular cell type in a sample or in a subject such as, for example, the number of monocytes or macrophages (i.e., M1 macrophages, M2 macrophages, etc.). Methods and techniques used to determining the amount or quantity or prevalence of a disclosed transcript or polypeptide or cell in a sample as used herein can refer to the steps that the skilled person would take to measure or ascertain some quantifiable value of the transcript or polypeptide or cell in the sample. The art is familiar with the ways to measure an amount of a disclosed nucleotides, transcripts, polypeptides, cells, etc. In an aspect, “determining” can refer to a rate of some type of activity, such as, for example, the rate of or extent of polarization of monocytes into M1 macrophage or M2 macrophages.

As used herein, “pro-inflammatory cytokines” can include but are not limited to cytokines characterized as being inducible and belonging to different families, including IL-1, IL-6, and TNFα. In an aspect, pro-inflammatory cytokines can include subsequently discovered molecules of the TNF family with related properties that promote inflammatory host reactions such as secreted lymphotoxin a (also known as TNFβ), the heterotrimeric membrane-bound lymphotoxin αβ, LIGHT, CD40 ligand, Fas ligand, CD30 ligand, CD27 ligand, 4-1BB ligand, and the Ox40 ligand. Other pro-inflammatory cytokines can include IFN, IL-2, both products chiefly of TH1 cells as, well as IFN.

As used herein, “anti-inflammatory cytokines” can include, but are not limited to, interleukin (IL)-1 receptor antagonist (IL-1ra), IL-4, IL-6, IL-10, IL-11, and IL-13, or other cytokines known to those of skill in the art.

As used herein, the term “level” refers to the amount of a target molecule or a target cell in a sample, e.g., a sample from a subject. The amount of the molecule or the cell can be determined by any method known in the art and will depend in part on the nature of the molecule (i.e., gene, mRNA, cDNA, protein, enzyme, etc.) or the nature of the cell type (M1 or M2 macrophage, monocyte, etc.). The art is familiar with quantification methods for nucleotides (e.g., genes, cDNA, mRNA, etc.) as well as proteins, polypeptides, enzymes, etc. The art is familiar with quantification methods for various cell types. It is understood that the amount or level of a molecule or the amount or level of a cell type in a sample need not be determined in absolute terms, but can be determined in relative terms (e.g., when compare to a control or a sham or an untreated sample).

As used herein, the terms “administering” and “administration” refer to any method of providing a pharmaceutical preparation to a subject. Such methods are well known to those skilled in the art and include, but are not limited to, local administration, oral administration, transdermal administration, intradermal administration, intraperitoneal administration, intrathecal administration, intraocular administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically, that is, administered to treat an existing disease or condition. In further various aspects, a preparation can be administered prophylactically, that is, administered for prevention of a disease or condition.

As used herein, “infiltrating” can refer to the accumulation or concentration of a particular cell type at a particular location within a subject. For example, in an aspect, the cell type can be monocytes. In an aspect, the particular location within a subject can be the site of an injury or disease progression, such as the heart following myocardial infarction or in the atherosclerotic heart.

By “modulate” is meant to alter, i.e., by an increase or a decrease. As used herein, a “modulator” can mean a composition that can either increase or decrease the expression level or activity level of a gene or gene product such as a peptide, or that can either increase or decrease the prevalence of a specific cell type such as a monocyte, a M1 macrophage, or a M2 macrophage. Modulation in expression or activity does not have to be complete. For example, expression or activity can be modulated by about 10%, 20%, 30%, 40%, 50%, 60%, 70%, 80%, 90%, 95%, 99%, 100% or any percentage in between as compared to a control cell wherein the expression or activity of a gene or gene product has not been modulated by a composition, such as, for example, a composition comprising BMP-7 or M-CSF or a second agent that polarize monocytes or a combination thereof. Similarly, modulation of the prevalence of a specific cell type does not have to be complete. For example, prevalence of a specific cell type can be modulated by about 10%, 20%, 30%, 40%, 50%, 60%, 70%, 80%, 90%, 95%, 99%, 100% or any percentage in between as compared to a control cell or tissue or subject, wherein the cell or tissue or subject has not been modulated by a composition, such as, for example, a composition comprising BMP-7 or at least one second agent that polarize monocytes or M-CSF or a combination thereof. In an aspect, “modulate” can refer to increasing or decreasing the rate or extent of some type of activity, such as, for example, the increasing or decreasing the rate or the extent of polarization of monocytes into M1 macrophage or M2 macrophages.

As used herein, the terms “effective amount” and “amount effective” can refer to an amount that is sufficient to achieve the desired result such as, for example, the polarization of monocytes to M1 macrophages and/or M2 macrophages. As used herein, the terms “effective amount” and “amount effective” can refer to an amount that is sufficient to achieve the desired an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, then the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

Disclosed are the components to be used to prepare a composition of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

In an aspect, “polarization” as used herein can be used to refer to the functional and spatial asymmetry that plays a role for cells such as immune cells, which undergo chemotaxis and sense extracellular stimulus gradients. For example, distinct subsets of effector T-cells that bridge the innate and adaptive immune system are said to be polarized in the sense that these cells are characterized by the expression of distinct subsets of cytokines and receptors. For examples see: Be1 cells, Be2 cells, IL17-Th2 cells, Th1 cells, Th2 cells, Th3 cells, Th5 cells, Th6 cells, Th9 cells, Th17 cells, Th22 cells. For a comprehensive gene expression profile of Th1 cells and Th2 cells, see Nagai et al., 2001.

In an aspect, “differentiation” as used herein can be used to refer to cellular differentiation, which can be used to describe the process by which a less specialized cell becomes a more specialized cell type. For example, differentiation can occur numerous times during the development of a multicellular organism as the organism changes from a simple zygote to a complex system of tissues and cell types. Differentiation can be considered a common process in adults as well: adult stem cells divide and create fully differentiated daughter cells during tissue repair and during normal cell turnover. Differentiation can dramatically change a cell's size, shape, membrane potential, metabolic activity, and responsiveness to signals.

The present invention can be understood more readily by reference to the following detailed description of the invention and the Examples included therein.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Preliminary Experiments

Effect of BMP-7 on Monocyte Cell Differentiation

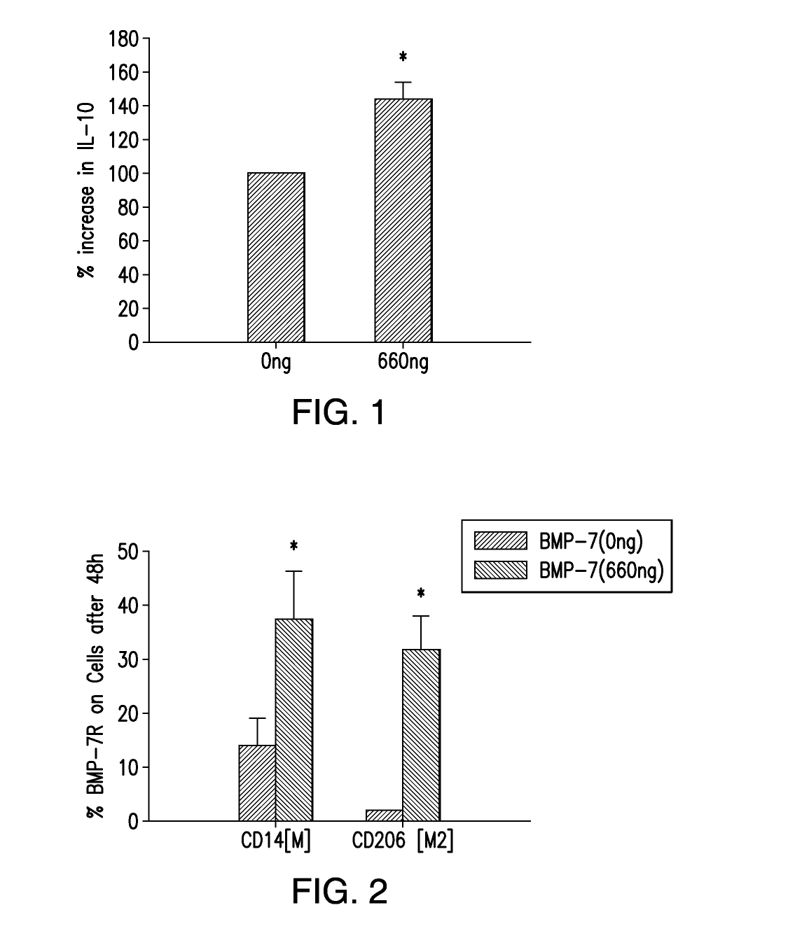

THP-1 monocytes were obtained from ATCC and maintained as reported (Daigneault et al., 2010). A BMP-7 dose curve was established by treating THP-1 cells for 48 hrs. with 0 ng, 165 ng, 330 ng, 660 ng and 1320 ng/mL of BMP-7. The data indicated that at the 660 ng/mL dose, the expression level of IL-10, an anti-inflammatory cytokine released by M2 macrophages, was significantly increased when compared with controls (FIG. 1-group treated with 660 ng of BMP-7 compared with group treated with 0 ng—*p<0.0 and n=6). These data indicate that the monocytes, following exposure to BMP-7, differentiated into M2 macrophages.

Whether the THP-1 cells contained a receptor for BMP-7 and whether the differentiating macrophages released markers for M2 macrophages was next examined. The immunostaining and confocal imaging data indicated that THP-1 cells expressed a type 2 receptor (BMP2R) for BMP-7. Following exposure to BMP-7, the number of THP-1 cells that expressed BMPR2 was significantly increased. These THP-1 cells were immunostained with the CD206 antibody, which is an antibody specific for M2 macrophages. These data showed an increased number of cells that co-stained for BMPR2 and CD206 (FIG. 2). Confocal imaging with a merged image for CD14, CD206, and BMPR2 staining showed that monocytes having BMPR2 were in the process of polarizing into M2 macrophages. FIG. 2 provides a quantitative analysis of percentage of cells containing the BMP-7 receptor following 48 hrs of treatment with 660 ng of BMP-7 compared to 0 ng. A significant increase in expression of BMP-7 receptor was found in monocytes and M2 macrophages when treated with 660 ng. (*p<0.05 vs. BMP-7 (0 ng)).

Establishment of Atherosclerosis Model

As ApoE KO mice are prone to develop ATH, these mice are a widely used animal model to generate ATH (Nam et al., 2009; Nam et al., 2010). The PLCA model is associated with ˜10%-15% mortality in ApoE KO. A partial left coronary artery ligation (PLCA) model in ApoE KO mice was used to develop ATH. Male and female ApoE KO mice between 8 to 10 weeks old were purchased and segregated into three groups: Sham (n=6), PLCA (n=6), and PLCA+BMP-7 (n=6). PLCA surgery was performed as described in Nam et al., 2009 and Nam et al., 2010.

Effects of BMP-7 on Plaque Formation

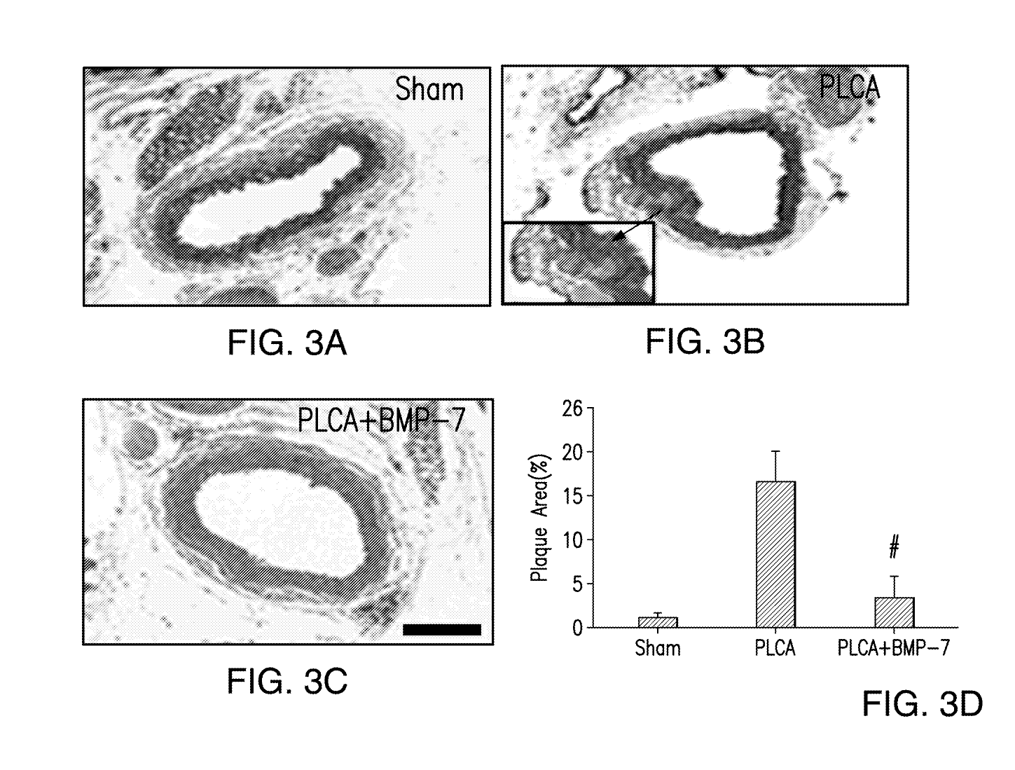

Following surgery, animals were injected intravenously (i.v.) with either mouse recombinant BMP-7 (200 μg/kg (Sekiya et al., 2009; Zeisberg et al., 2003) or saline. Injections were given for two additional days after surgery. Following the last injection, mice were left to recover for 14 days. After 14 days, blood velocity function was recorded using echocardiography. Coronary arteries were isolated and embedded in paraffin blocks as described in (Zeisberg et al., 2003; Singla et al., 2007; Singla et al., 2011; Singla et al., 2011). Sections were used to verify the presence of plaques or the presence of a complete occlusion. Cross sections were stained with Hematoxylin and Eosin (H&E) and the plaque area was measured under a bright field light. The histochemical staining data showed the formation of plaque in the PLCA group, which was significantly increased when compared with sham controls. The formation of plaque was significantly reduced in the BMP-7 treated groups (FIG. 3A-FIG. 3C; scale=100 μm). In FIG. 3B, the arrow indicates enlarged area of increased plaque formation in the PLCA group. In FIG. 3D, the histogram shows quantitative analysis of the percentage of plaque area with the PLCA+BMP-7 group, which showed showing significantly less percentage of plaque area as compared to the PLCA group (#p<0.05 vs. PLCA). FIG. 3B-FIG. 3C show photomicrographs of tissue sections stained with H&E displaying the plaque area following PLCA.

Effects of BMP-7 on Monocyte Infiltration Post-PLCA

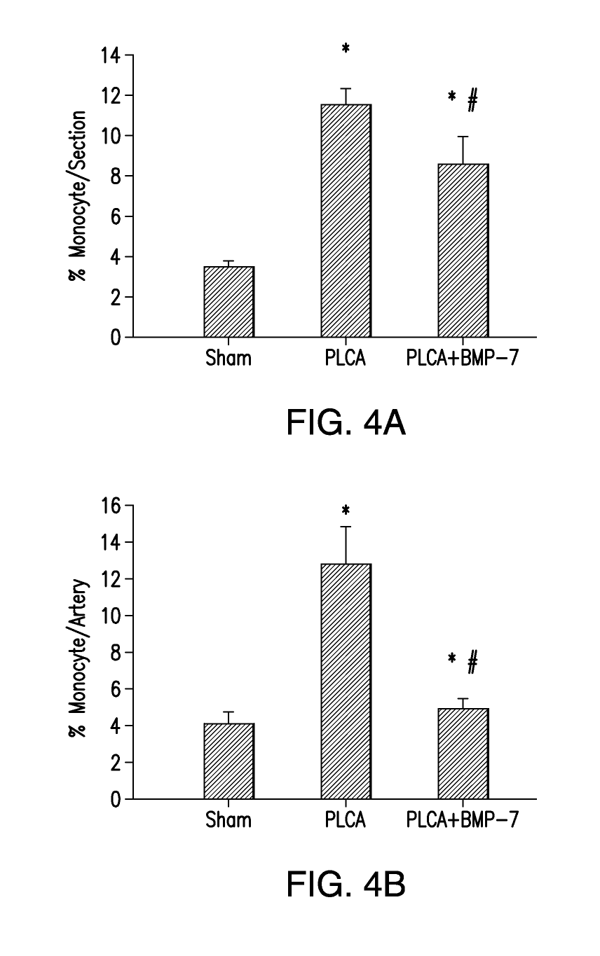

To examine monocyte infiltration, mouse-specific CD14 antibodies were used to identify monocytes. Confocal microscopy images specific for monocyte identification in and around the artery were obtained. The quantitative data indicate a significant increase in monocytes in the PLCA group. Exposure to BMP-7 inhibited this increase. FIG. 4A shows quantitative analysis of percentage of monocytes in sections. FIG. 4B shows quantitative analysis of percentage of monocytes in arteries in sections. (*p<0.05 vs. Sham; #p<0.05 vs. PLCA).

Effects of BMP-7 on Monocyte Polarization

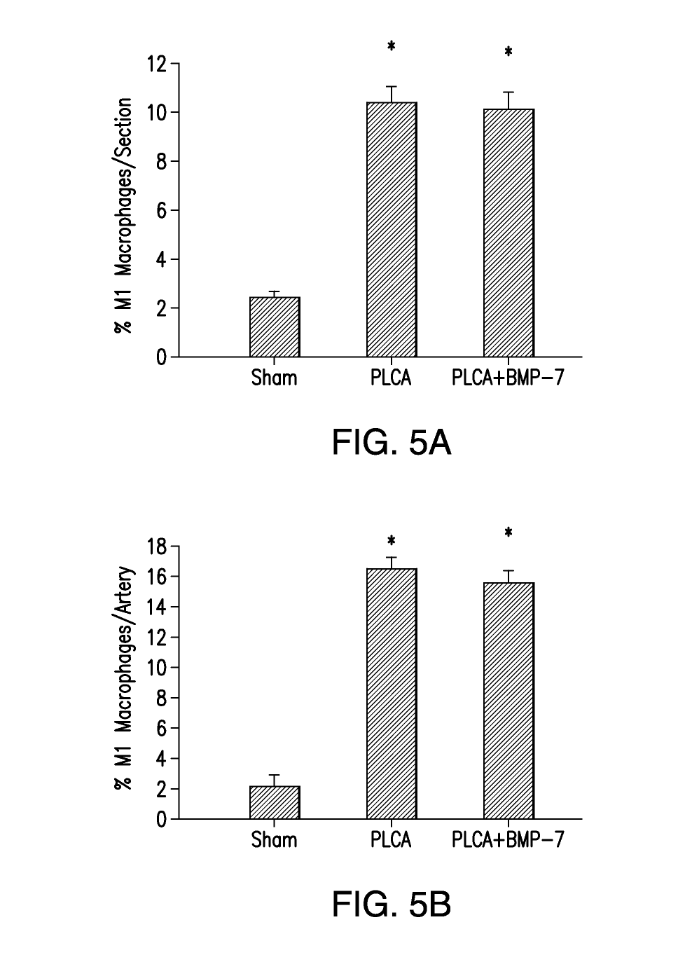

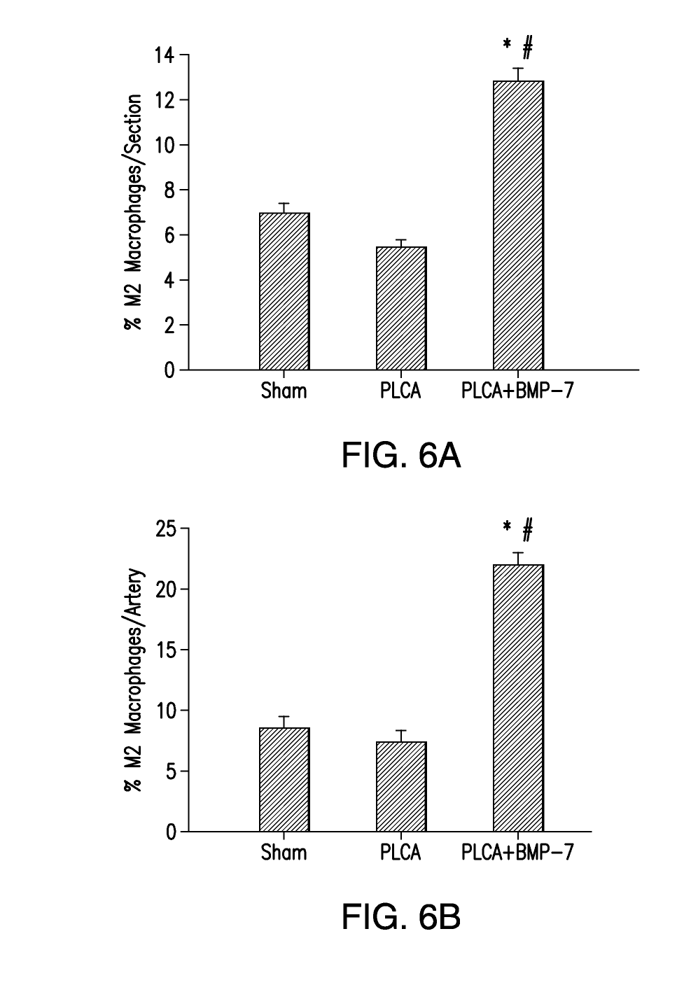

To determine the effects of BMP-7 on monocyte polarization into M1 and M2 macrophages, M1 macrophage-specific antibody staining with CD11C and M2 macrophage-specific antibody staining with CD206 was performed. Confocal images of M1 macrophages stained for CD11C antibody were obtained. Confocal images of CD206 antibody immunostaining specific for M2 macrophages were also obtained. The quantitative data for M1 macrophages showed a significant increase in M1 macrophages post-PLCA in ApoE KO mice. Exposure to BMP-7 showed no effects on the M1 polarized macrophages, which indicated that BMP-7 had no effects on M1 macrophage differentiation following PLCA (FIG. 5A-FIG. 5B). FIG. 5A shows quantitative analysis of percentage of M1 macrophage in sections. FIG. 5B shows quantitative analysis of percentage of M1 macrophage in arteries in sections. (*p<0.05 vs. Sham). Whereas the percentage of M2 macrophages in the PLCA group was lower than that of the control group (FIG. 6), the percentage of M2 macrophages in the PLCA+BMP-7 group was significantly increased. This finding indicated that BMP-7 played a role in the polarization of monocytes into M2 macrophages (FIG. 6). FIG. 6A shows quantitative analysis of percentage of M2 macrophage in sections. FIG. 6B shows quantitative analysis of percentage of M2 macrophage in arteries in sections. (*p<0.05 vs. Sham and PLCA).

Determination of BMP-7 Type II Receptor (BMP-7R2 or BMPR2) on Monocytes and Co-Localization with M2 Macrophages

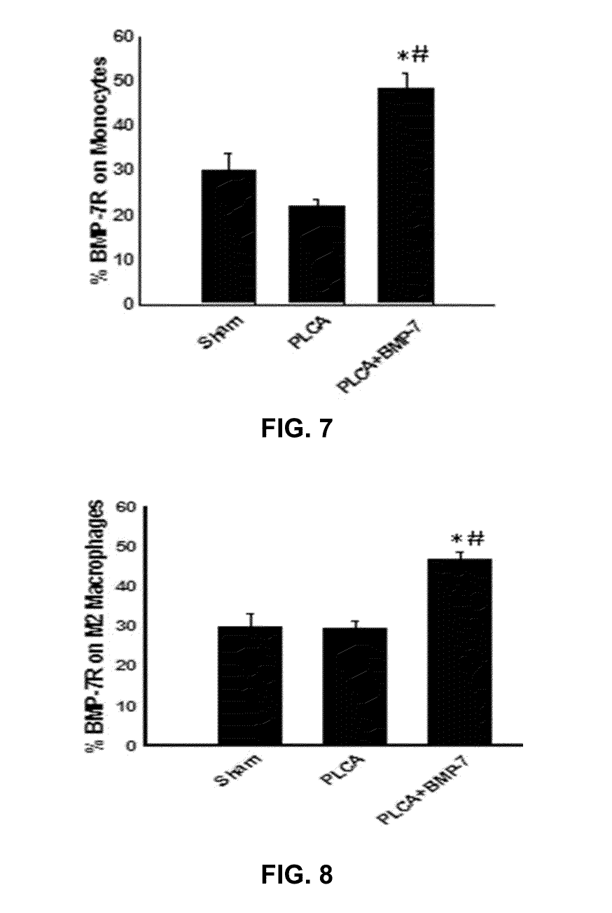

BMPR2 was identified on THP-1 monocytes and on M2 macrophages. Whether infiltrating monocytes in vivo also contained BMPR2 was also determined. Sections were stained with anti-BMPR2 antibody. Sections were also co-stained with the monocyte-specific antibody CD14. BMPR2 staining was evident in the artery area, which co-localized with the monocyte-specific CD14 antibody. The merged photomicrographs confirmed co-localized staining for both BMPR2 and monocytes. The percentage of monocytes containing BMPR2 was also determined. The presence of BMPR2 in the groups treated with BMP-7 was significantly up regulated as compared with PLCA group. This indicated that BMP-7 played a role in the monocyte activation for its receptor and then polarized specifically into M2 macrophages. In FIG. 7 shows the percentage of BMP-7R2 on monocytes. (*#p<0.05 vs. Sham and vs. PLCA).

As the PLCA+BMP-7 group experienced an increase in the number of M2 macrophages in the coronary artery, whether these M2 macrophages had any relationship with BMPR2 was examined. Double staining was performed using an antibody against BMPR2 and the M2 macrophage specific antibody CD206. The confocal data and the quantitative analysis indicated that a percentage of M2 macrophages also contained a BMPR2. BMPR2 was highly up regulated in the BMP-7 treated groups. These data indicated that monocytes containing BMPR2 polarized into M2 macrophages that still expressed BMPR2. FIG. 8 shows the percentage of BMPR2 on M2 macrophages. (*#p<0.05 vs. Sham and vs. PLCA).

Effects of BMP-7 on Levels of Anti-Inflammatory Cytokines

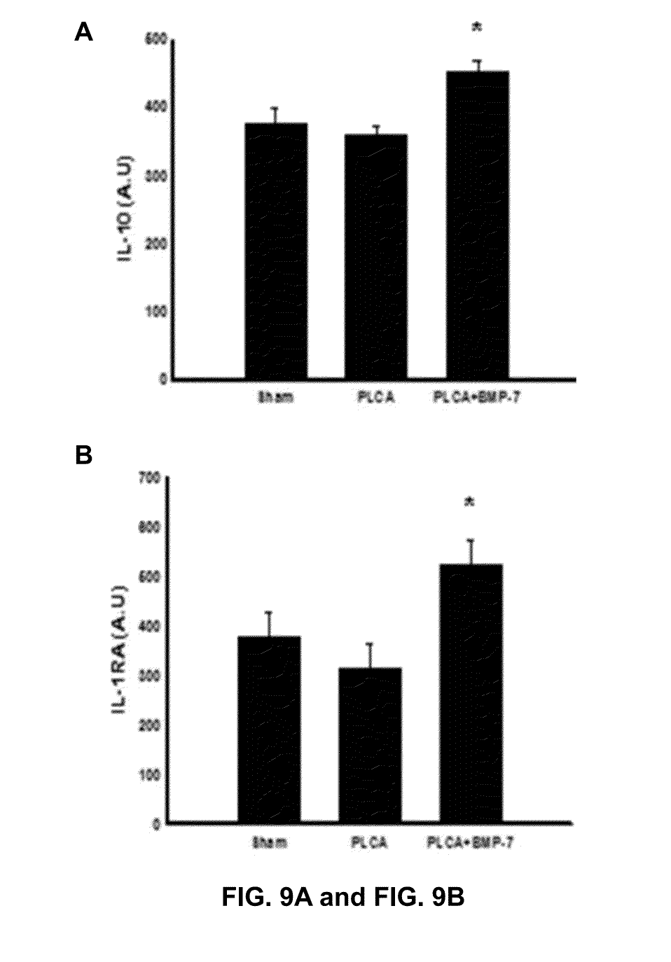

After determination of increased M2 macrophages following BMP-7 treatment, the levels of the anti-inflammatory cytokines IL-10 and IL-1ra was examined. IL-10 and IL-1ra are released by M2 macrophages. These data showed that the levels of IL-10 and IL-1ra in PLCA groups were not significantly decreased when compared with the sham controls. However, following BMP-7 treatment, the levels of these cytokines were significantly upregulated, indicating that BMP-7 significantly increased the level of M2 macrophages, which in turn, released anti-inflammatory cytokines (FIG. 9). In FIG. 9A-FIG. 9B, blood plasma was removed from each group and prepared for ELISA analysis of IL-10 and IL-1 receptor antagonist (IL-1ra). FIG. 9A shows IL-10 and FIG. 9B show IL-1ra released cytokine (*p<0.05 vs. sham and PLCA; n=4-5 animals per group).

Determination of BMP-7 Levels in Blood Circulation

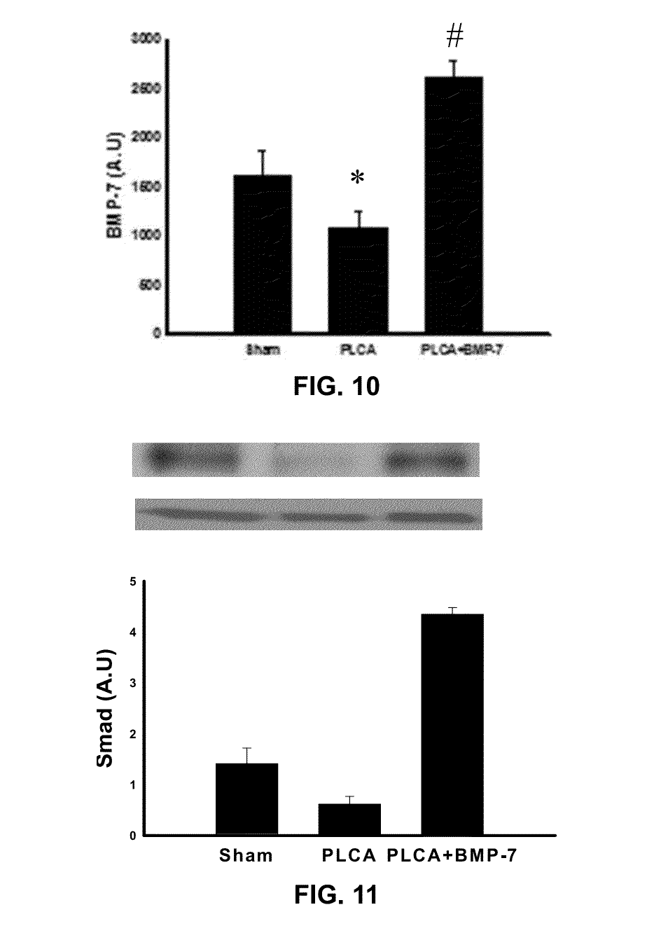

Whether BMP-7 was still present in the blood circulation two weeks after the last injection of BMP-7 was determined. ELISA data showed that BMP-7 in the blood circulation was decreased following surgery. However, two weeks after the last injection of BMP-7, the levels of BMP-7 remained high, which indicated that BMP-7 is required for the polarization of monocytes into M2 macrophages (FIG. 10). FIG. 10 shows that blood plasma shows levels of BMP-7 were confirmed using ELISA. BMP-7 was significantly lower in the PLCA group; however, BMP-7 was increased in the PLCA+BMP-7 group. (*p<0.05 vs. Sham, #p<0.05 vs. sham and vs. PLCA, n=4-5 per group).

Effects of BMP-7 on SMAD Pathway

Whether the SMAD-1, SMAD-5, and SMAD-8 pathways were involved in BMP-7 mediated monocyte polarization into M2 macrophages was examined. Homogenates were prepared from coronary arteries and proteins were estimated. SDS-PAGE and Western blotting was performed using previously described methods (Glass et al., 2011; Singla et al., 2008). FIG. 11 shows that levels of phosphorylated SMADs were lower compared with sham controls. Following treatment with BMP-7, an increased level of phosphorylated SMADs was observed. The increased M2 polarized macrophages were associated with increased levels of phosphorylated SMADs, indicating that the SMAD pathway was involved in the polarization of monocytes into M2 macrophages. In FIG. 11, BMP-7 injection increased SMAD activity following PLCA ligation. Western blot (top panel) shows increased expression of SMAD in the PLCA+BMP-7 group and a decrease was observed in the PLCA group as compared with the Sham group. In FIG. 11, the histogram (bottom panel) shows a quantitative analysis. (n=2-3 animals per group).

Echocardiography Measurements of the Left Common Carotid Artery Velocity

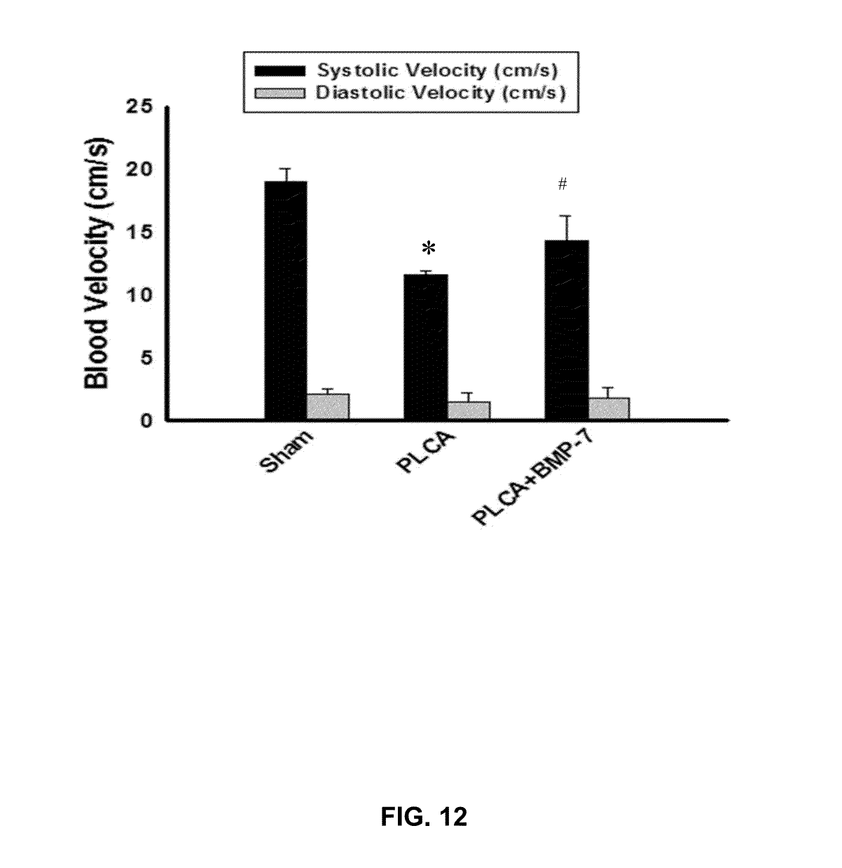

Fourteen (14) days after PLCA surgery, a Phillips SONOS 5500 Ultrasound system was used to assess the left common carotid artery internal systolic and diastolic velocities. Briefly, mice were anesthetized, the neck hair was removed, and mice were positioned in a supine direction. Under vascular settings, three to four 2D images were recorded and analyzed on the machine. Both internal common carotid artery systolic (SV-cm/s) and diastolic blood velocity (DV-cm/s) measurements were obtained. The data showed that the systolic blood velocity in the PLCA group was significantly reduced when compared with the sham control, which indicated that these mice had developed ATH. The PLCA+BMP-7 treated group showed increased systolic blood velocity in the left carotid artery, which indicated that BMP-7 conferred beneficial effects at the functional level (FIG. 12). In FIG. 12, the histogram shows a significant increase in systolic velocity in the PLCA+BMP-7 group as compared to the PLCA group. The PLCA group shows decreased numbers as per control. #p<0.05 vs. PLCA; *p<0.05 vs. Sham).

Experiment 1

To characterize the effects of BMP-7 on monocytes polarizing into M2 macrophages, a stress-induced microenvironment in monocytes (human acute monocytic leukemia cell line THP-1) was established to simulate injury. The data provided herein demonstrate that monocytes (THP-1 cells) contain a BMP type II receptor (BMPR2), and that the number of BMPR2 is significantly increased following treatment with BMP-7. BMP-7 treatment also increased the number of M2 macrophages and BMPR2. The increase in the numbers of M2 macrophages was associated with increased levels of anti-inflammatory cytokines, whereas levels of pro-inflammatory cytokines were decreased following BMP-7 treatments.

Cell Culture

THP-1 cells, a monocyte cell line, and Roswell Park Memorial Institute medium (RPMI 1640) were purchased from ATCC and were supplemented with 10% fetal bovine serum and 0.05 mmol/L beta-mercaptoethanol. THP-1 cells were grown in suspension in 25 cm² flasks as per ATCC's recommendations.

Treatment with BMP-7

THP-1 cells were treated with 0 ng, 165 ng, 330 ng, 660 ng, and 1320 ng of BMP-7 (Bioclone, California, USA). In a 48-well plate, 20,000 total cells were plated per well for 24 hr. After 24 hr, the same doses of BMP-7 were added for an additional 48 hr. Supernatant from the well plates was collected. To optimize the dose for BMP-7, the supernatant was used to induce the release of the anti-inflammatory cytokine IL-10, which was an indicator of M2 polarized macrophages. These experiments were conducted in duplicate.

Preparation of Monocyte Polarized M2 Macrophage Cell Culture Model

To delineate the effects of BMP-7 (both with and without stress conditions), a cell culture model system was generated. To induce stress conditions, a specialized apoptotic cell culture model using H9c2 cells (cardiomyocytes) was used to generate an apoptotic-conditioned medium (ACM). The ACM was used to treat THP-1 cells. In brief, H9c2 cells were cultured and maintained as reported in Singla et al., 2007. A 60 mm (diameter) tissue culture plate was plated with 5×105 cells for 24 hr. Next, apoptosis was induced with 400 μmol/L of H₂O₂ for 2 hr. After 2 hr, the ACM was removed from the plate and filtered to be used for further studies. The THP-1 cells were then divided among the following six (6) groups to measure: (1) the expression of BMPR2, (2) the number of polarized M2 macrophages, and (3) the release of pro-inflammatory and anti-inflammatory cytokines. These six (6) groups were: (i) control, THP-1 cells (20,000 cells/well in a 48-well plate); (ii) THP-1 cells+BMP-7; (iii) THP-1 cells+ACM (40% v/v); (iv) THP-1 cells+ACM+BMP-7; (v) THP-1 cells+ACM+BMP-7+follistatin (500 ng/mL; a BMP-7 inhibitor); and (vi) THP-1 cells+follistatin (which served as control for the inhibition of endogenous BMP-7).

Immunocytochemistry

After 48 hr of treatment, cells were centrifuged, the supernatant was removed, and the cell pellet was prepared. The cell pellet was used to prepare a cell smear on a Plus slide. The smears were air dried, fixed with 4% paraformaldehyde, and permeabilized with 0.3% Triton X-100 at room temperature. Blocking was performed using 10% normal goat serum non-specific antibody. At room temperature, slides were incubated with anti-CD-14 antibody (a marker for human monocytes), anti-CD-206 antibody (Abcam; a marker for M2 macrophages), and anti-BMPR2 (Santa Cruz, USA). Slides were washed and incubated with secondary antibodies goat anti-mouse 568 and or anti-goat FITC. The slides were then mounted with vectashield mounting medium containing total nuclei stain DAPI and examined under Olympus and confocal microscopy.

Pro-Inflammatory and Anti-Inflammatory Cytokines

Enzyme-linked immunoassay (ELISA) kits (RayBiotech, USA) were used to examine the levels of pro-inflammatory cytokines (interleukin 6 (IL-6), monocyte chemotactic protein-1 (MCP-1), and tumor necrosis factor α (TNFα)), as well as the levels of anti-inflammatory cytokines (IL-10 and IL-1ra). In brief, cell supernatant from all groups was collected and loaded into pre-coated 8-well strips for 2.5 hr at room temperature. After washing, the detection antibody (biotinylated anti-mouse antibodies) was added followed by HRP-conjugated streptavidin and TMB One-Step substrate reagent. When the blue color developed, stop solution was added and the wells were measured at 450 nm with the microtiter plate reader (BioRad). Graphs were plotted as percent change, after analysis of the data obtained from the ELISA readings as arbitrary units (A.U.).

Statistical Analysis

Data were calculated and expressed as the mean±SE. Statistical significance was calculated at p<0.05 using both one way ANOVA and Tukey's test.

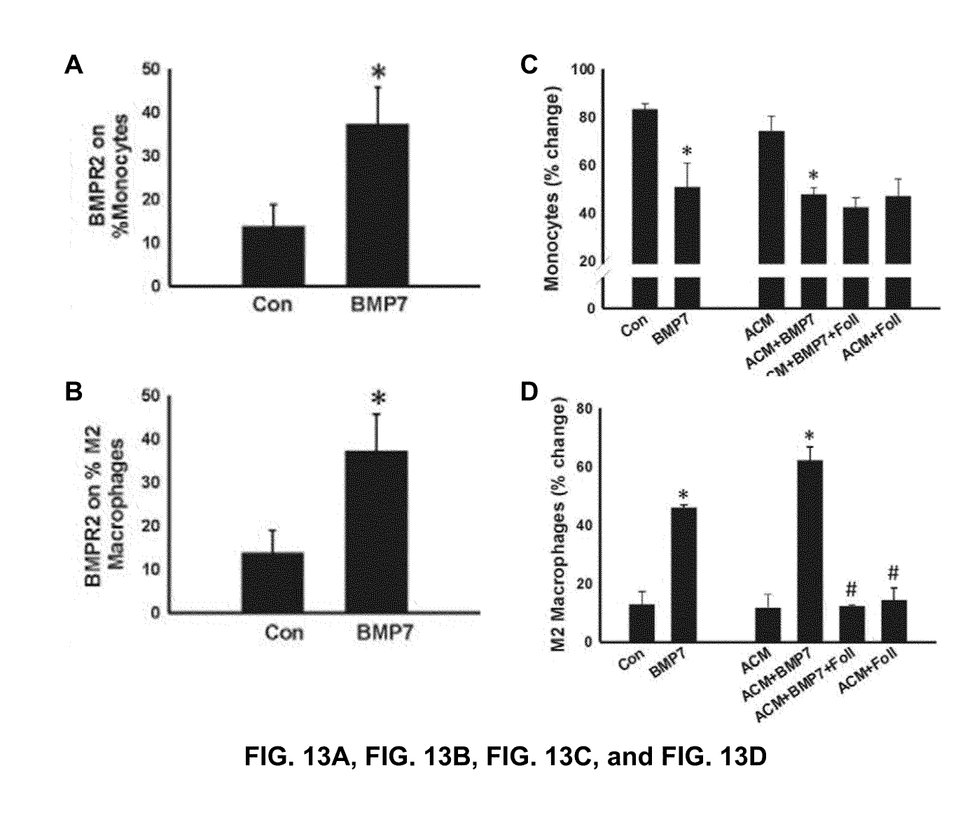

A BMP-7 dose curve was established by treating THP-1 cells for 48 hr with 0 ng/mL, 165 ng/mL, 330 ng/mL, 660 ng/mL, or 1320 ng/mL of BMP-7. These data indicated that 660 ng/mL was the optimal dose. This dose significantly increased expression levels of IL-10, an anti-inflammatory cytokine released by M2 macrophages. Subsequently studies utilized 660 ng/mL dose. The immunostaining and confocal data indicated that THP-1 cells, which were confirmed with monocyte marker CD14 staining, also expressed BMPR2. Quantitative data showed that the number of THP-1 cells that also positively stained for BMPR2 was significantly increased following BMP-7 treatment (FIG. 13A). Quantitative data showed that the number of CD206 positive M2 macrophages that positively stained for BMPR2 was also increased. For example, FIG. 13A and FIG. 13B show the quantitative analysis of the percentage of monocytes and macrophages containing BMPR2 following 48 hr of treatment with 660 ng of BMP-7 compared with 0 ng (*p<0.05 compared with the control (0 ng BMP-7)). FIG. 13C provides the quantitative analysis of the percentage of monocytes, and FIG. 13D provides the percentage of M2 macrophages present following 48 hr of treatment for each group (*p<0.05 compared with the control; #p=non-significant compared with the ACM control).

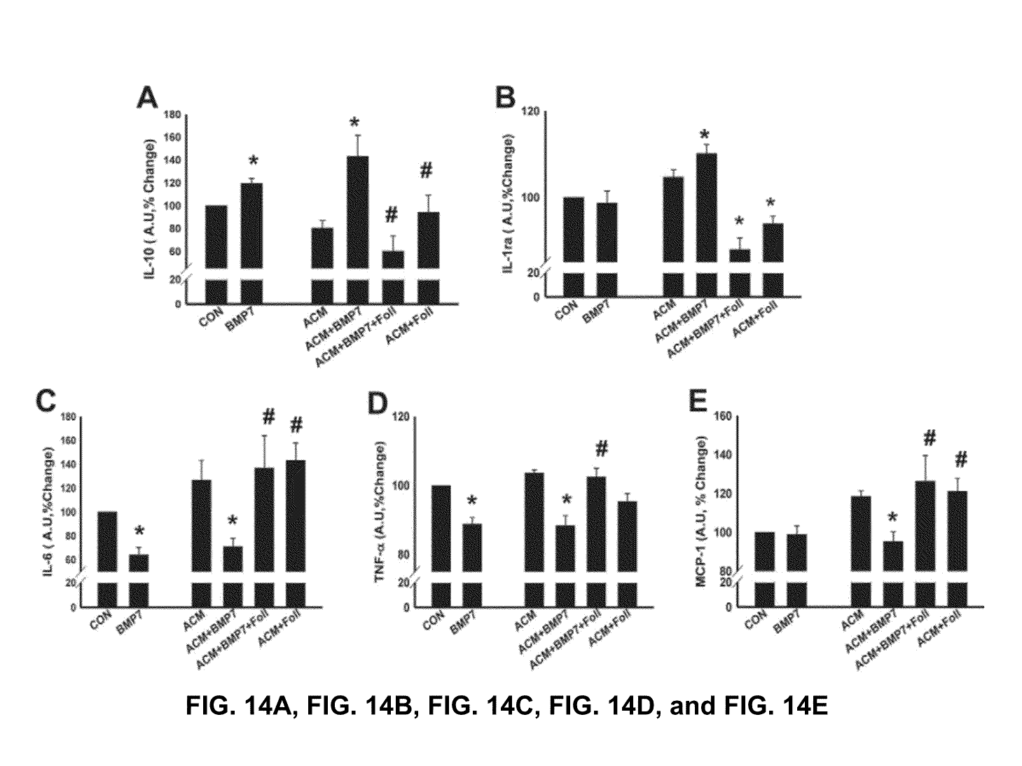

The THP-1 cells treated with BMP-7 demonstrated an increase in the levels of anti-inflammatory cytokine IL-10 but did not demonstrate an increase in the level of IL-1ra (FIG. 14A and FIG. 14B). When these cells were treated with ACM to generate stress conditions, the levels of anti-inflammatory cytokines IL-10 and IL-1ra were significantly increased (p<0.05) as compared to the respective controls (FIG. 14A and FIG. 14B). Moreover, treatment with follistatin, an inhibitor for BMP-7, showed a significant decrease (p<0.05) in IL-10 and IL-1ra levels, indicating that BMP-7 polarized THP-1 cells into M2 macrophages.

FIG. 14 shows that BMP-7 treatment increased anti-inflammatory cytokine activity and decreased pro-inflammatory cytokine activity. FIG. 14A and FIG. 14B show quantitative analysis of interleukin 10 (IL-10) and IL-1ra activity, which are anti-inflammatory cytokines expressed by M2 macrophages. FIG. 14C, FIG. 14D, and FIG. 14E respectively show quantitative analysis of IL-6, tumor necrosis factor α (TNF-α), and monocyte chemotactic protein-1 (MCP-1), which are pro-inflammatory cytokines. These data were generated from samples taken from each group following 48 hr of treatment (n=4-6 duplicates;*p<0.05 compared with the respective control cells; and #p=non-significant compared with the control cells).

Whether BMP-7 had any effect on the levels of pro-inflammatory cytokines IL-6, TNF-a, and MCP-1, which play a major role in the pathogenesis of inflammatory disease progression was examined. The data indicated that BMP-7 treatment, with or without ACM-treated THP-1 cells, resulted in significantly decreased levels of IL-6 and TNF-a when compared with respective controls. (FIG. 14C, FIG. 14D, and FIG. 14E). However, the levels of MCP-1 in untreated THP-1 cells (control), and the levels in THP-1 cells treated with BMP-7 were not different. BMP-7 treatment in the ACM-treated THP-1 cells resulted in levels of MCP-1 that were significantly reduced when compared with the non-treated THP-1+ACM-treated cells, indicating that stress conditions have a unique role in the regulation of pro-inflammatory cytokine MCP-1. Moreover, the increased levels of anti-inflammatory cytokines and decreased levels of pro-inflammatory cytokines in THP-1 cells following BMP-7 treatment were reversed when the cells were treated with the BMP-7 inhibitor follistatin. These data indicate that BMP-7 altered the expression of pro-inflammatory and anti-inflammatory cytokines

These data indicate that BMP-7 treated THP-1 cells (monocytes) demonstrated increased expression of BMPR2 in both monocytes and M2 macrophages (as evidenced by co-localization of antibodies against CD14 and BMPR2 and co-localization of antibodies against CD206 and BMPR2). These data also show that BMP-7 treatment decreased the number of monocytes in cell culture, which decrease was accompanied by a concomitant increase in M2 macrophages (as evidenced by co-localization of CD14 and CD206 immunostaining). The data showed that there was no significant difference in the percent change in the monocytes following BMP-7 treatment and treatment with both ACM+BMP-7. However, the ACM+BMP-7 treatment demonstrated an increased number of M2 macrophages as compared to the level of M2 macrophages resulting from THP-1 cells treated with only BMP-7. This finding indicates that stress conditions exert an effect on M2 macrophage polarization.

The data also indicated that, when compared to the controls, the levels of pro-inflammatory IL-6 and TNF-α were significantly reduced with or without ACM-treated BMP-7 groups. Following treatment with BMP-7, the decreased levels of pro-inflammatory cytokines correlated with decreased levels of monocytes. BMP-7 did not demonstrate any effects on pro-inflammatory cytokine MCP-1. However, when cells were treated with ACM+BMP-7, the reduction in levels of MCP-1 was evident.

Here, follistatin was used to understand the effect of BMP-7 on the polarization of monocytes into M2 macrophages and on the level of released cytokines. The data indicated that follistatin inhibited the BMP-7 effect on the polarization of M2 macrophages and inhibited the up-regulation of anti-inflammatory cytokines IL-10 and IL-1ra. However, follistatin had no effect on the percentage change of monocytes. The decrease in levels of pro-inflammatory cytokines IL-6, TNF-a, and MCP-1 was reversed with follistatin.

Experiment 2

To characterize the role of BMP-7 during the development of ATH in ApoE-deficient animals, these experiments utilize immunohistochemical and real-time PCR techniques to measure the expression levels of (i) pro-inflammatory and anti-inflammatory cytokines, (ii) other inflammatory molecules, and (iii) molecules specific for M1 and M2 phenotype macrophages. These techniques are further used to explore the relationship between BMP-7 and M1 macrophages and M2 types of macrophages.

Examine Effects of BMP-7 on ATH

A well-established ApoE KO mouse PLCA model is used to generate ATH. The PLCA model is associated with ˜10%-15% mortality in ApoE KO. There are 12-14 animals per group and animals are divided into three groups: (1) Sham, (2) PLCA, and (3) PLCA+BMP-7. On the day of surgery, BMP-7 (200 μg/kg; i.v.) is administered to the animals in the PLCA+BMP-7 group. Over the next two days, BMP-7 is continuously administered. Fourteen (14) days after PLCA, the animals are euthanized.

Additional experiments using the same dose (200 μg/kg; i.v.), but different time points, are performed. For example, animals are sacrificed 24 hours, 4 weeks, 12 weeks, and 16 weeks. For long-term experiments, an additional single dose of BMP-7 is given bi-monthly. To determine whether an additional dose is required, levels of BMP-7 in the blood are tested 2-3 weeks after each injection. If BMP-7 levels are approximately equal to or below control levels, one or more additional doses are given. After euthanization, coronary arteries are isolated and subjected to histological (H&E) and immunohistochemistry analysis. Moreover, coronary artery sections are analyzed using oil red staining to determine the effect of BMP-7 on fat deposition. Blood samples are collected to perform plasma cytokines and gene expression. Heart function is examined via echocardiography.

Determine Total Number of Monocytes and Macrophages Post-PLCA

To determine the infiltration and/or presence of monocytes and/or M1 and M2 macrophages following PLCA, serial sections containing plaque areas are immunostained using a mouse-specific CD14 antibody for monocytes (e.g., antibody from Abbiotec), a CD11 antibody for M1 macrophages (e.g., antibody from Abcam), and a CD206 antibody for M2 macrophages (e.g., antibody from Abcam). These data provide information regarding the various types of cell distribution at the base level. Confocal and fluorescence microscopy are used to acquire images and the NIH Image J program is used for analysis (Singla et al., 2007; Singla et al., 2011; Singla et al., 2011; Singla et al., 2008).

Identify Monocytes and Differentiated M1 and M2 Macrophages Post-PLCA

To determine the number of infiltrated monocyte cells that have started polarization into M1 macrophages or M2 macrophages, BrdU (50 mg/kg body weight, i.p. (Beltrami et al., 1997; Hosoda et al., 2009) is administered. BrdU identifies nuclei in S phase to label actively dividing cells. Active cell growth of infiltrated monocyte cells is identified using double-label immunostaining for monocyte specific marker CD14 and BrdU (Dako) antibodies. CD14 identifies infiltrated cells and BrdU labels DNA synthesizing cells. At a given time, the number of cells that have started cell division and cell type polarization is determined. Ki-67 is a nuclear antigen associated with cell division and labels proliferating cells (G1-, S-, G2-phase, and mitosis), but not in quiescent or resting cells (G0-phase). Double-label immunostaining are performed to detect proliferating cells using Ki-67 (Dako) antibody and monocyte infiltrated cells with CD14 antibodies.

Monocyte infiltration and polarization is examined. Over a course of time, monocytes are identified using the CD14 antibody. Next, cell differentiation (M1 vs. M2 macrophages) is determined using double-labeling immunostaining to detect monocytes that have been polarized into M1 and M2 macrophages. Sections are also stained for BMPR2 and combined with M1 or M2 macrophage cell-specific staining to determine whether monocytes containing BMPR2 have fully converted into specific phenotypes. If cells are positive with both stainings, then the cells are in the process of polarization. Sections are stained with respective FITC or rhodamine-conjugated secondary antibodies and counterstained with DAPI for nuclear visualization. The percentage of cells is calculated. The total number of positive cells specific for monocytes, M1 macrophages, or M2 macrophages are counted, and then divided by the total number of DAPI nuclei and multiplied by 100. Slides are analyzed using Olympus fluorescence and confocal microscopy.

Measure Levels of Pro-Inflammatory and Anti-Inflammatory Cytokines

A cytokine array ELISA (e.g., available from Ray Biotech) is used (i) to establish the presence of different cytokines and (ii) determine whether the cytokine profile correlates with the presence of one type of macrophage or another. For example, using commercially available kits from Ray Biotech and R&D Systems, the pro-inflammatory cytokines IL-6, TNF-α, and MCP-1 released by M1 macrophages and anti-inflammatory cytokines IL-10 and IL-1 receptor antagonist are measured in the blood plasma. (Singla et al., 2008; Glass et al., 2011).

Real-time PCR methods are used to determine the mRNA levels of these pro-inflammatory and anti-inflammatory cytokines in the macrophage-rich lesion area (Glass et al., 2011; Glass et al., 2011). Primers specific for these cytokines are designed using Applied Biosystems Primer Express software V2.0. Western blotting is used to detect and quantify alterations in the expression levels of the cytokines following exposure to BMP-7 (Glass et al., 2011; Glass et al., 2011).

Identify M1 and M2 Phenotype Macrophage-Specific Genes Affected by BMP-7

Using the macrophage-rich lesion area, RT-PCR is used for the isolation of cytoplasmic RNA (RNAeasy kit). The integrity of the RNA, which is later converted to cDNA, is determined using the Agilent Bioanalyzer (Singla et al., 2011; Glass et al., 2011). Per the manufacturer's instructions, commercially available Affymetrix arrays (Mouse Genome 430 2.0 Arrays, Affymetrix, CA) are used for whole genome expression array analysis. Appropriate controls are applied. Highly up-regulated or highly down-regulated specific genes following BMP-7 treatment is confirmed with real-time PCR and then optionally with ELISA or Western blotting if needed. Real-time PCR is used to measure expression of M1 macrophage-specific iNOS, IL-1β, and TNF-α gene expression. Expression of Arginase-1, which is specific for M2 macrophages, is also determined. As M-CSF and GM-CSF are involved in M1 and M2 macrophage polarization, the expression of these genes is measured with real time-PCR and subsequently confirmed at the protein level with Western blotting.

Functional Assessment of Coronary Artery Blood Flow

Following PCLA, echocardiography is performed on infarcted hearts at short-term (24 hrs. and 14 days) and long-term (4, 12, and 16 weeks) time points.

Experiment 3

To characterize the BMPR2 mediated effects of BMP-7 on M2 macrophages and on monocytes, a loss of function strategy is employed.

Determine Effect of BMP-7 on Disrupted BMPR2 Monocytes

Monocytes are isolated from two different sources: (1) bone marrow obtained from femurs, and (2) peripheral blood of 10-12 week old ApoE KO (Houthuys et al., 2010; Tamaki et al., 2008). Isolated monocytes are transfected with BMPR2 specific sequences, which are identified using the ABI system. Sequence-specific siRNA are generated and transfected into these cell populations. Disruption of BMPR2 is confirmed using RT-PCR and Western blot analysis. Selected cells are isolated and maintained in the cell culture system.

Cells are maintained in a standard cell culture medium that consists of DMEM supplemented with fetal calf serum and macrophage colony-stimulating factor (M-CSF). The effects of specific BMP-7 at 660 ng/mL are determined (Huse et al., 2011). The dosage can be altered. To establish the presence of M2 macrophages, cells are also treated with and without BMP-7 at various times up to 96 hrs. Also, untransfected monocytes are used with the BMP-7 antagonist follistatin, which forms a complex with BMP-7 protein and prevents binding to any type of BMP-7 receptor.

A series of experiments involve non-transfected monocytes. M-CSF is removed from the cell culture medium and then BMP-7 alone is added to the cells. The percentage of cells that can be polarized into M2 macrophages without M-CSF is assessed. Immunohistochemistry is performed. The levels of one or more pro-inflammatory and anti-inflammatory cytokines are determined. Gene and protein expression levels for one or more genes and/or protein products are measured.

Experiment 4

M-CSF is a unique growth factor responsible for M2 polarized macrophages from monocytes in a cell culture system (Martinez et al., 2006; Sierra-Filardi et al., 2010). The data presented herein demonstrated that M-CSF is another novel growth factor that plays a role in (i) the polarization of cultured THP-1 cells into M2 macrophages, and (ii) the polarization of infiltrating monocytes in ApoE KO mice. These experiments determine whether, during the polarization of monocytes into M2 macrophages, BMP-7 and M-CSF act independently of each other.

Determine the Effects of BMP-7 and M-CSF in M2 Macrophage Polarization In Vivo

Generation of heterozygous mice using the backcross method is known to the art (Babamusta et al., 2006; Qiao et al., 1997; Smith et al., 1995). In heterozygous (double KO) mice from op mice (mice specific for M-CSF deletion) and ApoE KO mice is generated as described in the art Babamusta et al., 2006; Qiao et al., 1997; Smith et al., 1995). The op male mice and ApoE KO female (Jackson Laboratories) are obtained and backcrossed 10 times. Genotyping is performed using standard PCR (Babamusta et al., 2006). Animals are housed in a specific pathogen-free (SPF) room. Animals are divided into one of four groups: (1) Sham, (2) PLCA, (3) PLCA+BMP-7, and (4) PLCA+BMP-7+M-CSF (12-14 animals per group). The PLCA+BMP+M-CSF group is utilized to determine whether M-CSF delivered intravenously restores monocyte function in heterozygous mice, and to determine whether both growth factors are necessary.

As BMP-7 can act via activin type II receptor, additional experiments comprising the knock down of activin type II receptor using siRNA methodology to see a complete loss of function are performed.

On the day of surgery, BMP-7 is injected (200 μg/kg; i.v.), and is also injected continuously over the next two days. Animals are euthanized at different times. 24 hrs. prior to the day of surgery, M-CSF (250 μg/kg; i.v., (Asakura et. al., 1997) is injected. M-CSF is also given at any other times that BMP-7 is given. Coronary artery sections are prepared. H&E and oil red stainings are performed to determine atherosclerosis and fat deposition. Echocardiography is used to determine blood velocity function.

Determine the Effects of BMP-7 and M-CSF in M2 Macrophage Polarization in Cell Culture System

These experiments probe the actions of BMP-7 and M-CSF in M2 macrophages. To understand the direct effects of BMP-7 and M-CSF in the cell culture model, cells are divided into four groups: (1) Control THP-1 cells, (2) THP-1+BMP-7, (3) THP-1 cells+BMP-7+Follistatin (a BMP-7 inhibitor (Iemura et al., 1998)), and (4) THP-1 cells+BMP-7+soluble form of M-CSF receptor or M-CSF specific antibodies (Kutza et al., 2000). Data are collected at different time points up to 96 hrs. M2 polarized macrophages are identified using immunostaining and secreted IL-10 levels. The levels of released M-CSF are determined using ELISA. Levels of gene expression and protein levels are established using methods known in the art. M2 macrophages are confirmed using immunohistochemistry, by measuring the levels of pro-inflammatory and anti-inflammatory cytokine, by measuring protein levels using Western blotting, and measuring gene expression

Experiment 4

ATH involves monocyte infiltration and the polarization of the monocytes into M1 or M2 macrophages. This polarization depends on the various cytokines and growth factors present in the developing plaque area (Wolfs et al., 2011; Kadl et al., 2010; Devaraj et al., 2011). These data provided herein show that BMP-7 enhances the polarization of monocytes into M2 macrophages. The role of BMP-7 in other diseases has been investigated, and BMP-7 upregulated SMAD-1, SMAD-5, and SMAD-8 (Moustakas et al., 2005; Sovershaev et al., 2011). BMP-7 was also implicated in the role of p38, ERK, and JNK pathways during osteoblast differentiation and mononuclear cells (Moustakas et al., 2005; Sovershaev et al., 2011).

Determine the Role of BMP-7 and Mechanisms of M2 Polarized Macrophages

The role of BMP-7 as well as the role of inhibitors of SMAD (Hasegawa et al., 2009) and MAP Kinase in the polarization of M2 macrophages in coronary arteries is determined (Singla et al., 2008; Bradley et al., 2008; Stebbins et al., 2008; Su et al., 2010). This is done in ApoE KO mice, heterozygous models, and cell culture systems.

PLCA is performed as described above. A cell culture model is also used as described above. Coronary arteries are isolated and homogenates are prepared (Glass et al., 2011; Singla et al., 2008). For cell culture experiments, cell lysates are prepared according to methods utilized in Singla et al., 2011. M2 macrophages are confirmed by using immunohistochemistry, by measuring the levels of pro-inflammatory and anti-inflammatory cytokine, by measuring protein levels using Western blotting, and by measuring gene expression using techniques known to those skilled in the art.

SDS-PAGE and Western blot analyses are performed to determine the protein expression levels of SMADs, p38, ERK, and JNK (antibodies are available from, for example, Santa Cruz Biotechnology/Cell Signaling) (Singla et al., 2008; Bradley et al., 2008; Stebbins et al., 2008; Su et al., 2010). Both the total level as well as the level of phosphorylated proteins are measured. (Singla et al., 2008; Bradley et al., 2008; Stebbins et al., 2008; Su et al., 2010). Densitometry is used to measure band density. ELISA is performed with different kits (e.g., from Sigma and Ray Biotech Inc.) to measure p38, ERK, and JNK in the coronary arteries and cell culture models. To determine the role of SMADs, p38, ERK, and JNK in M2 macrophages, animals are given selective inhibitors of SMADs (HSc025 15 mg/kg), p38 (SB203580, 50 mg/kg; see Su et al., 2010; Derynck et al., 2003-10 mg/kg has no effect on phosphorylated p38 inhibition and 100 mg/kg is toxic), ERK1/2 (PD98059 20 μg/kg i.p.), and JNK (BI-78D3 25 mg/kg) pathways (Hasegawa et al., 2009; Bradley et al., 2008; Stebbins et al., 2008; Su et al., 2010; Derynck et al., 2003). A cell culture dose for each inhibitor is also established.

Determine Whether M-CSF Induced M2 Polarized Macrophages also Involve SMAD Pathway

Whether BMP-7 and M-CSF share a common mechanistic pathway for the polarization of M2 macrophages is investigated. BMP-7 acts via the SMAD pathway in mononuclear cells and M-CSF activates the Ras pathway in osteoclast cells (Bradley et al., 2008). Using THP-1 cells to establish the pathway, specific inhibitors are utilized (HSc025 for SMAD, GW5074 for Ras, LY294002 for PI3K and Wortmannin) (Bradley et al., 2008). Activation and inhibition of these pathways is confirmed by ELISA and Western blot methods.

Determine if Inhibition of SMAD or Ras Pathways Influence M2 Polarized Macrophages

Whether the inhibition of a BMP-7 or M-CSF specific signaling pathway affects the polarization of M2 macrophages is explored. Specific inhibitors for both SMAD and Ras pathways in are used as follows: (1) THP-1 cells, (2) THP-1 cells+HSc025 (SMAD pathway inhibitor), and (3) THP-1 cells+GW5074 (Ras inhibitor). M2 macrophages are confirmed using immunohistochemistry, by measuring the levels of pro- and anti-inflammatory cytokine, by measuring protein levels using Western blotting, and by measuring gene expression using techniques known to those skilled in the art. The levels of various pathway-specific proteins are determined using ELISA and Western blot methods.

Experiment 5

Whether BMP-7 treatment caused M2 macrophage polarization via the activation of the PI3K-Akt-mTOR pathway was examined. The data provided herein demonstrated that following treatment with BMP-7, expression of p-SMAD-1, p-SMAD-5, and p-SMAD-8 significantly increased resulting in increased expression and activation of p-PI3K. Activation of PI3K lead to downstream activation of p-Akt and p-mTOR, following the initiation of the PI3K-Akt-mTOR pathway. Follistatin, a BMP-7 inhibitor, significantly reduced M2 macrophage polarization as well as PI3K-Akt-mTOR pathway suggesting its role in polarization. Additionally, LY-294002, an inhibitor of the PI3K pathway, was studied and corroborated the effects of BMP-7 on M2 macrophage polarization through the examination of M2 macrophage released anti-inflammatory cytokines, IL-10 and arginase-1.

In Vitro Cell Culture Model

As per manufacturer's instructions, human monocytes (THP-1 cells) purchased from ATCC, were grown in 25 cm² flasks and cultured every other day in RPMI 1640 (ATCC) supplemented with 10% fetal bovine serum and 0.05 mM β-mercaptoethanol (Rocher et al., 2012).

Stress Induced Injury Model

Apoptotic conditioned media (ACM) was generated and cell concentration used for monocytes was previously reported (Rocher et al., 2012; Singla et al., 2007). In brief, 60 mm tissue culture dishes were each plated with 5×10⁵ H9c2 cardiomyoblasts for 24 hours. The cells were then treated with 400 μM H₂O₂ for 2 hours. Following treatment, media was collected, filtered, labeled ACM, and stored for future use.

Monocyte Polarization Treatment

Monocytes were divided into the following groups: Control (monocytes cultured in normal media), ACM (monocytes cultured in normal media+ACM (40% v/v)), ACM+BMP-7 (monocytes cultured in normal media+ACM (40% v/v)+660 ng/mL BMP-7), and ACM+BMP-7+Follistatin (Foll) (monocytes cultured in normal media+ACM (40% v/v)+660 ng/mL BMP-7+500 ng/mL Foll). Experimental conditions and mouse BMP-7 (Bioclone, San Diego, Calif.) and follistatin (Sino Biological, Beijing, China) concentrations were used as reported in Rocher et al., 2012. Briefly, monocytes were initially plated in a 24 well plate with 40,000 cells per well for 24 hours for stabilization. The cells were then treated based on the aforementioned groups for an additional 48 hours.

Ly-294002 Treatment

The PI3K inhibitor, LY-294002, was used as previously reported in Singla et al., 2008. The monocytes were divided into the following groups: Control (monocytes cultured in normal media), ACM (monocytes cultured in normal media+ACM (40% v/v)), ACM+BMP-7 (monocytes cultured in normal media+ACM (40% v/v)+660 ng/mL BMP-7), and ACM+BMP-7+LY) (monocytes cultured in normal media+ACM (40% v/v)+660 ng/mL BMP-7+40 μM LY). The monocytes were plated in a 24 well plate with 40,000 cells per well for 24 hrs. Then, the cells were treated according to the above groups for 48 hrs and next used for experimental analysis.

Immunohistochemistry of BMPR2 and Macrophage Markers

Cells were removed from the 24 well plate, centrifuged and used to make smears on ColorFrost Plus glass slides (Rocher et al., 2012). The smears were fixed with 4% paraformaldehyde and blocked with either 10% normal goat serum or normal donkey serum (Vector Labs) for 1 hr at room temperature. Following blocking, the smears were incubated with primary antibodies for either anti-CD14, anti-iNOS, anti-CD206, anti-arginase-1, or anti-BMPR2 overnight at 4° C. Next, appropriate secondary antibodies, goat anti-rabbit Alexa 568 or donkey anti-goat Alexa 488, were added for 1 hr at room temperature. Finally, the smears were stained with DAPI (4′,6-diamidino-2-phenylindole) (Vector Labs) and cover-slipped. The slides were analyzed under an Olympus fluorescent microscope.

Pro-Inflammatory and Anti-Inflammatory Cytokine ELISAs

ELISA kits were purchased from Raybiotech and used to quantify anti-inflammatory (IL-10) and pro-inflammatory (IL-6, TNF-α, and MCP-1) cytokine expression. Following treatment, the culture media was isolated from each group, centrifuged, and the supernatant collected. The supernatant was then added to pre-coated strips and the ELISAs were performed as per manufacturer's instructions and as reported in Rocher et al., 2012.

Western Blot Analysis

Western blot was performed as reported in Singla et al., 2008. In brief, cells were collected, centrifuged, and lysed on ice for 30 minutes in RIPA buffer supplemented with protease inhibitor cocktail, PMSF, sodium orthovanadate and sodium fluoride. Protein concentration was estimated for each cell lysate using the Bio-Rad protein assay and read on a microplate reader at 595 nm. Following protein concentration determination, 100 μg of the prepared protein samples were loaded and run on an 8 or 10% sodium-dodecyl sulfate polyacrylamide gel at 150V for 60 to 80 minutes depending on the molecular weight of the proteins to be separated. Next, proteins were transferred onto a PVDF membrane (Bio-Rad) which was then blocked with 5% milk and incubated with the primary antibodies p-SMAD1/5/8 (Cell Signaling, 1:1000), p-PI3K (Cell Signaling, 1:1000), Total PI3K (Cell Signaling, 1:1000), p-Akt (Cell Signaling, 1:1000), p-PTEN (Cell Signaling, 1:1000), p-mTOR (Cell Signaling, 1:1000), or Total mTOR (Cell Signaling, 1:1000). Following primary antibody incubation, membranes were washed, incubated with secondary antibody anti-rabbit IgG (Cell Signaling, 1:1000), and then exposed with a Visualizer. Densitometry analysis was completed with the help of Image J software which allows for the quantification of band intensity. Developed films were scanned and opened using Image J software. A rectangle was placed on each band and the band intensity was analyzed as well as the background intensity. Quantification was determined by subtracting band intensity with background intensity. Protein expression was corrected with a loading control such as β-actin, total mTOR or total PI3K and this by dividing the protein densitometry value by its control. All western blot data is presented as: Protein densitometry/control protein densitometry.

Statistical Analysis

Data was analyzed using One-way analysis of variance (ANOVA) followed by the Tukey Test. Data is presented as a mean±SEM and statistical significance assigned when p-value<0.05.

Expression of BMPR2 Increased Following Treatment with BMP-7 on Monocytes

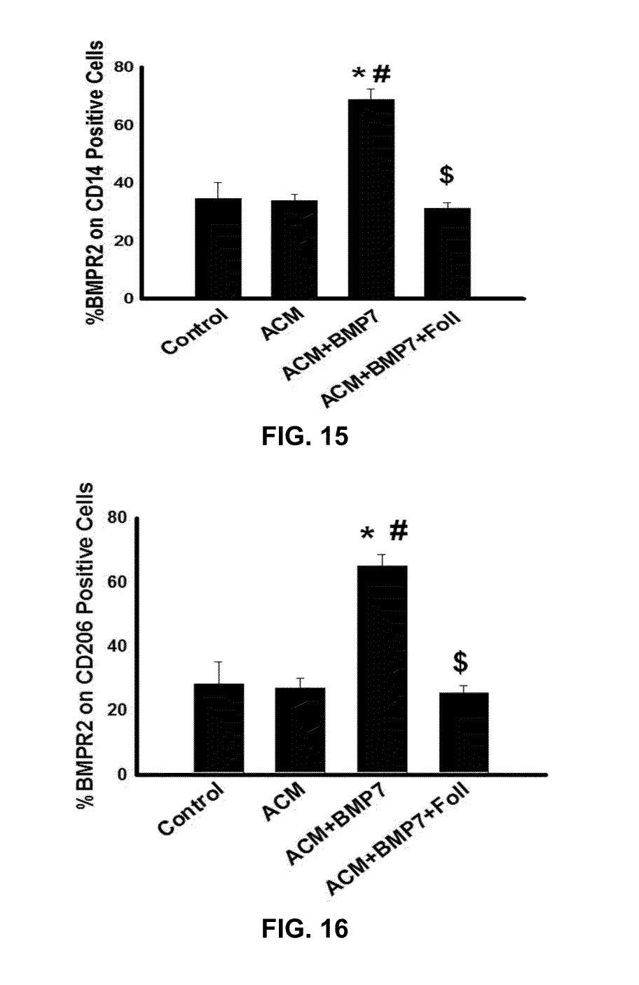

FIG. 15 demonstrated that following treatment with BMP-7, monocytic BMPR2 expression was significantly enhanced compared to that of the control and ACM groups. Additionally, BMPR2 expression on CD14⁺ cells was significantly inhibited in the ACM+BMP-7+Foll group when compared to the ACM+BMP-7 group, which indicated that follistatin blocked BMPR2 upregulation through inhibition of normal BMP-7 mediation. In FIG. 15, the following apply: *p<0.05 vs. Control, #p<0.05 vs. ACM, $ p<0.05 vs. ACM+BMP-7, n=6.

Expression of BMPR2 Increased Following Treatment with BMP-7 on M2 Macrophages

FIG. 16 shows that BMPR2 expression was significantly enhanced on polarized M2 macrophages in the ACM+BMP-7 group relative to the control and ACM groups. A significant reduction in BMPR2 expression on CD206⁺ M2 macrophages was observed in the ACM+BMP-7+Foll group when compared with the ACM+BMP-7 group. In FIG. 16, the following apply: *p<0.05 vs. Control, #p<0.05 vs. ACM, $p<0.05 vs. ACM+BMP-7, n=5-6.

Treatment with BMP-7 Decreased Expression of Pro-Inflammatory Markers

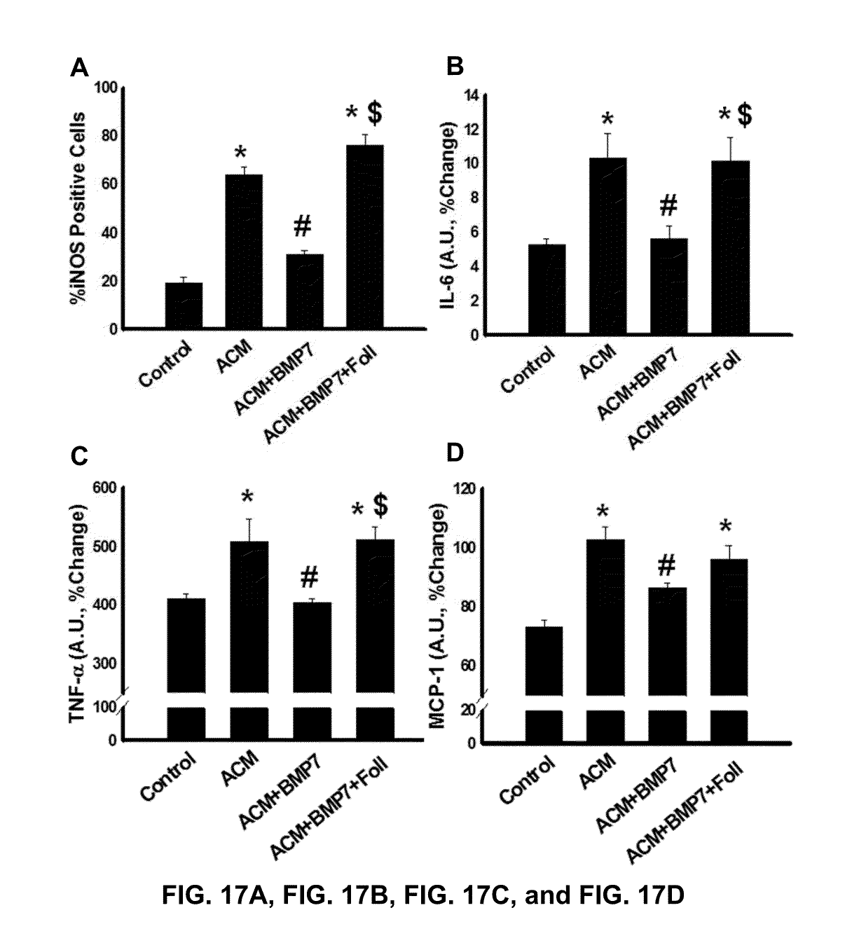

FIG. 17A shows that iNOS positive M1 macrophage concentrations were significantly enhanced in the ACM and ACM+BMP-7+Foll groups when compared to the control group (n=5-6). However, the number of M1 macrophages was significantly decreased in the BMP-7 treated group relative to the ACM treated group (FIG. 17A). When compared with the ACM+BMP-7 treatment group, M1 macrophages were significantly enhanced in the ACM+BMP-7+Foll group (FIG. 17A), indicating that BMP-7 prevented monocyte to M1 macrophage polarization.

Quantitative analysis of pro-inflammatory cytokine data is presented for IL-6 (FIG. 17B, n=5-6), TNF-α (FIG. 17C, n=4-6), and MCP-1 (FIG. 17D, n=5-7). There was a significant increase in IL-6, TNF-α, and MCP-1 expression in the ACM group when compared with the control group (FIG. 17B-FIG. 17D). This increased concentration of pro-inflammatory cytokines was significantly abolished following BMP-7 treatment and follistatin treatment resulted in significantly increased expression of IL-6, TNF-α, and MCP-1 (FIG. 17B-FIG. 17C). In FIG. 17A-FIG. 17D, *p<0.05 vs. Control, #p<0.05 vs. ACM, $p<0.05 vs. ACM+BMP-7.

Anti-Inflammatory Cytokine Expression Increased Following Treatment with BMP-7

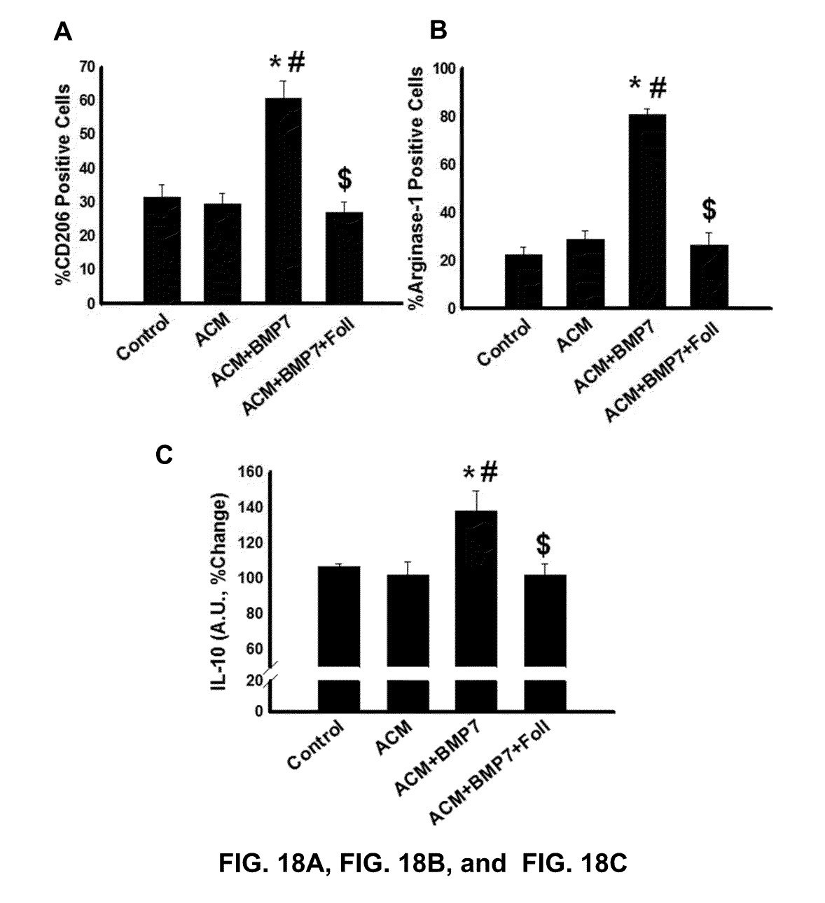

A quantitative analysis of the percentage of cells positive for the M2 macrophage marker, CD206, revealed a significant increase in the number of M2 macrophages in the ACM+BMP-7 group when compared to the control and ACM groups (FIG. 18A). However, the number of M2 macrophages was significantly decreased in the ACM+BMP-7+Foll group relative to the ACM+BMP-7 group (FIG. 18A, n=5-6).

Anti-inflammatory signaling molecules including arginase-1 and IL-10 were also assessed. Following treatment with BMP-7, a significant increase in the percentage of cells positive for arginase-1 was observed (FIG. 18B, n=5-6). Following follistatin treatment, a significant decreased was detected in the percentage of arginase-1 positive cells when compared to the ACM+BMP-7 group (FIG. 18B). Levels of IL-10 were significantly increased in the ACM+BMP-7 group when compared to the control and ACM groups (FIG. 18C, n=5-6). Treatment with follistatin abrogated the increased IL-10 expression following BMP-7 treatment (FIG. 18C). These data indicate that BMP-7 promoted M2 macrophage polarization and anti-inflammatory expression. In FIG. 18A-FIG. 18C, the following apply: *p<0.05 vs. Control, #p<0.05 vs. ACM, $p<0.05 vs. ACM+BMP-7.

Treatment with LY-294002 Inhibited the BMP-7 Induced Increase in IL-10 and Arginase-1

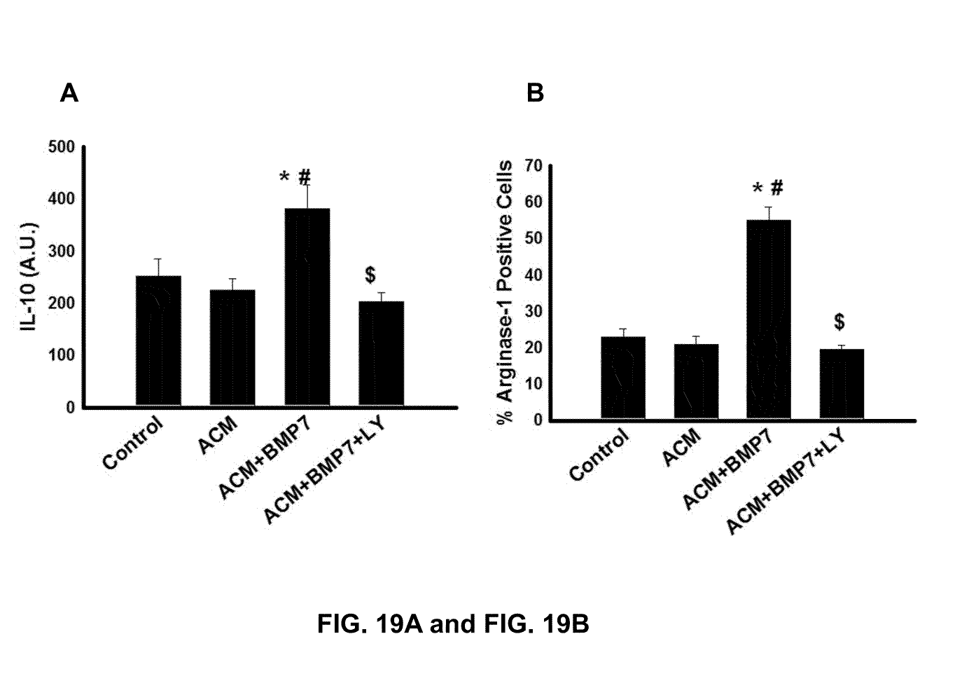

To confirm the role of BMP-7 on M2 macrophage anti-inflammatory cytokine expression through activation of the PI3K pathway, an inhibitor of the PI3K pathway-LY-294002 (LY), was used. FIG. 19A shows quantitative expression of anti-inflammatory cytokine, IL-10. Following treatment, the level of IL-10 remained the same between the Control and ACM groups; however, the level of IL-10 was significantly increased in the ACM+BMP-7 group when compared to the control and ACM groups (FIG. 19A). When treated with LY, the increased IL-10 expression significantly decreased compared to the ACM+BMP-7 group (FIG. 19A). The percentage of arginase-1 positive cells was also examined. FIG. 19B shows that there was no significant increase in percentage of positive cells in the Control and ACM groups. But when treated with BMP-7, the percentage of arginase-1 positive cells significantly increased. Following LY treatment, the percentage of arginase-1 positive cells significantly decreased when compared to the ACM+BMP-7 group (FIG. 19B). These data indicate that treatment with a PI3K inhibitor such as LY decreased expression of anti-inflammatory cytokines which were released by M2 macrophages. In FIG. 19A-FIG. 19B, the following apply: *p<0.05 vs. Control, #p<0.05 vs. ACM, $p<0.05 vs. ACM+BMP-7, n=5-7.

BMP-7 Promoted Activation of SMAD1/5/8 and PI3K

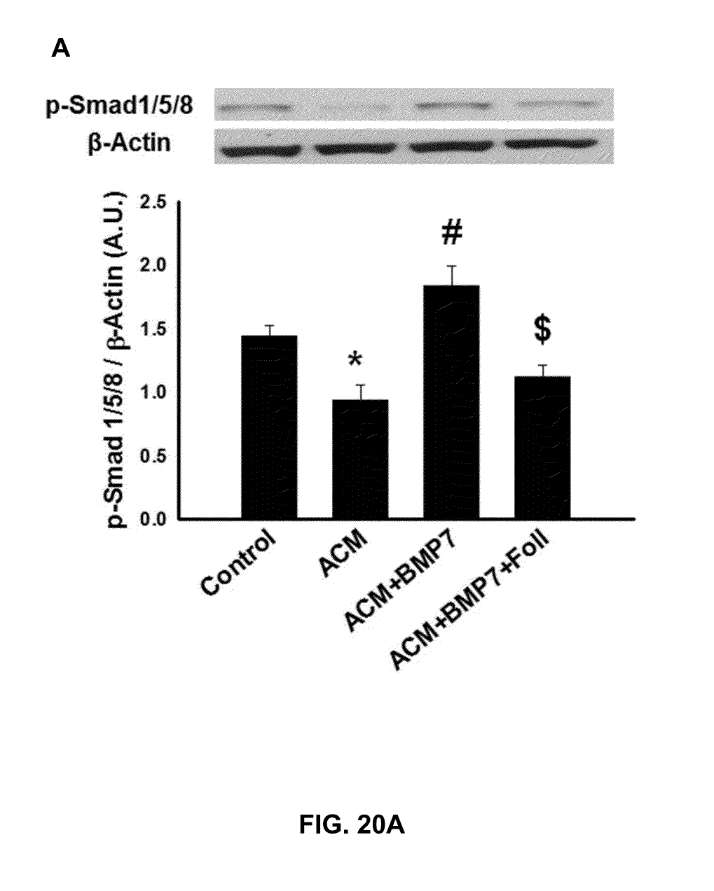

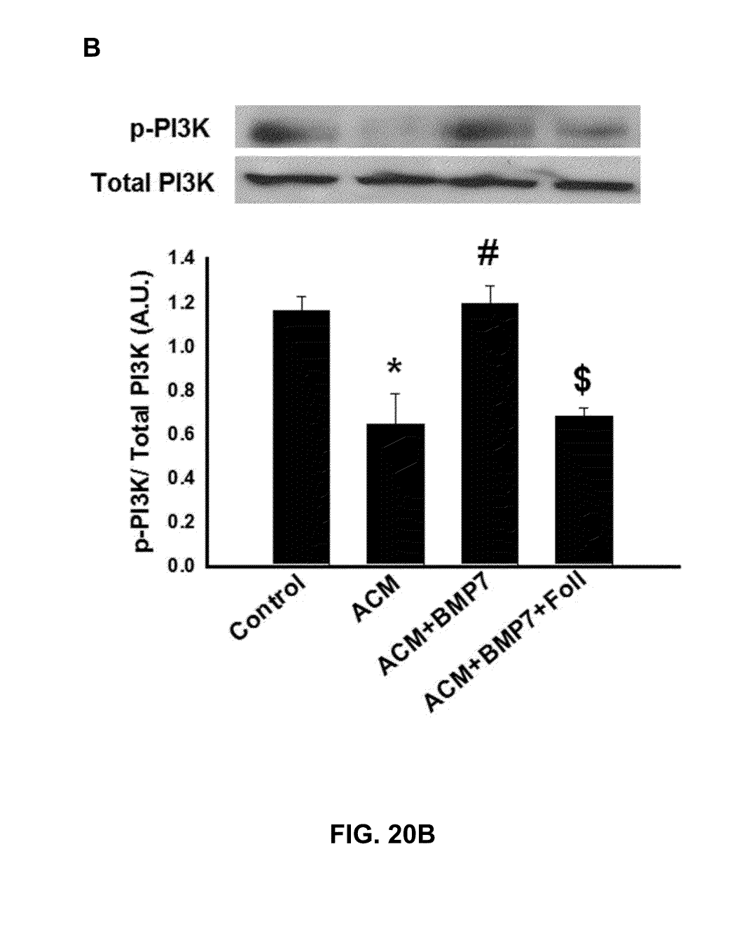

To elucidate mechanisms by which BMP-7 promoted M2 macrophage polarization, mediators of BMP signaling were investigated. FIG. 20A (top panel) depicts representative activated SMAD-1, SMAD-5, and SMAD-8, downstream effectors of BMP signaling, and corresponding control β-actin blots. Quantitative analysis indicated a significant decrease in activated SMAD-1, SMAD-5, and SMAD-8 expression in the ACM group (FIG. 20A (bottom panel)). Following BMP-7 treatment, phosphorylated Smad1/5/8 (p-Smad1/5/8) expression increased, an effect that was abolished following treatment with follistatin (FIG. 20A).

Representative blots of phosphorylated phosphoinositide 3-kinase (p-PI3K), a enzymatic signaling molecule responsible for promoting cell proliferation, differentiation, and survival, and total PI3K, are shown in the top panel of FIG. 20B. Activated PI3K expression was significantly decreased in the ACM group when compared to the control group. p-PI3K expression was significantly increased following BMP-7 treatment and activated PI3K was significantly decreased in the ACM+BMP-7+Foll group when compared to the ACM+BMP-7 group (FIG. 20B). In FIG. 20A-FIG. 20B, the following apply: *p<0.05 vs. Control, #p<0.05 vs. ACM, $p<0.05 vs. ACM+BMP-7, n=5-7.

Downstream Activation of Akt and mTOR Occurred Following BMP-7 Treatment

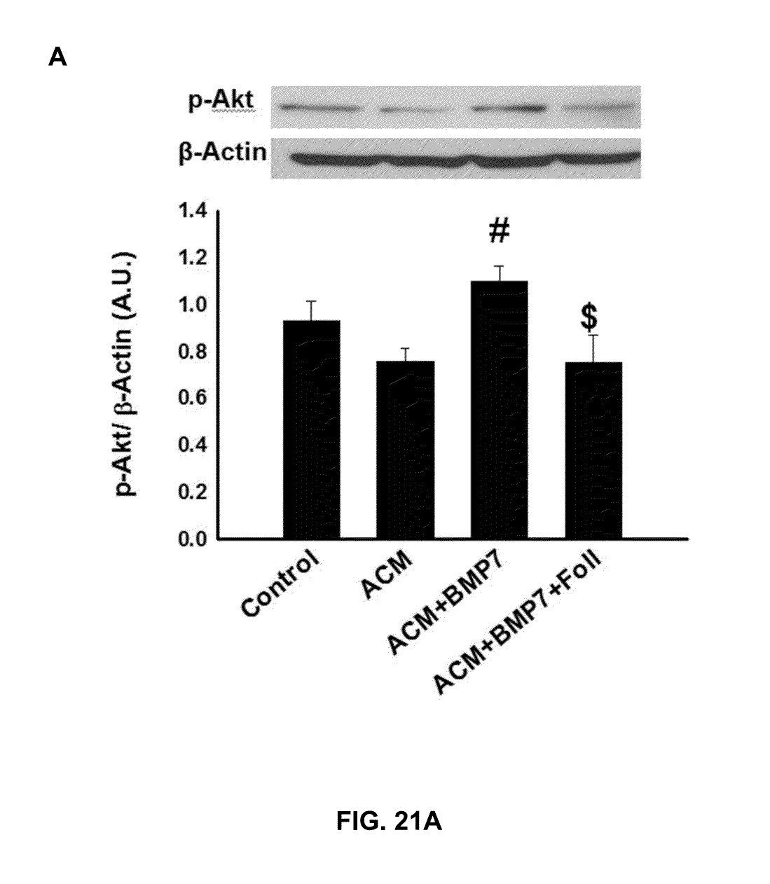

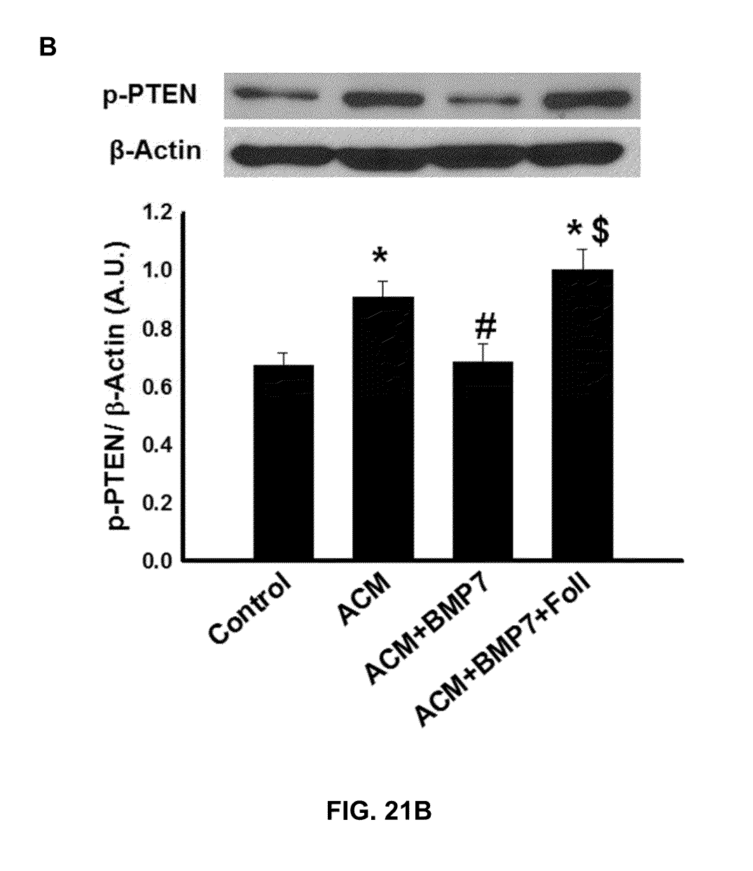

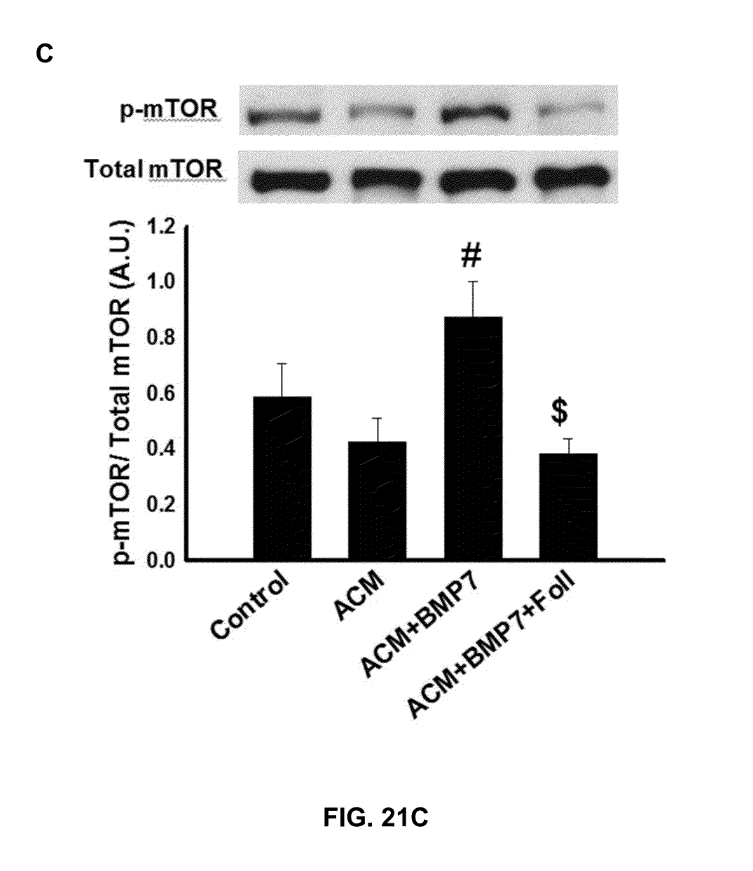

Downstream mediators of the PI3K pathway were investigated to discern mechanisms of PI3K pathway augmentation via BMP-7. FIG. 21A-FIG. 21C (top panels) show representative blots of phosphorylated Akt (p-Akt) (FIG. 21A), phosphatase and tensin homolog (p-PTEN) (FIG. 21B), and mammalian target of rapamycin (p-mTOR) (FIG. 21C). Activated Akt was significantly enhanced in the ACM+BMP-7 group and a significant decrease in p-Akt expression was observed in the ACM+BMP-7+Foll group (FIG. 21A). PTEN, an inhibitor of PI3K and subsequent Akt activation, was significantly upregulated in the ACM and ACM+BMP-7+Foll groups when compared to the control group (FIG. 21B). However, treatment with BMP-7 inhibited PTEN activation (FIG. 21C). Densitometric analysis of p-mTOR over total mTOR expression revealed a significant increase in expression in the ACM+BMP-7 group when compared to the ACM group, which was abolished following follistatin treatment (FIG. 21C). In FIG. 21A-FIG. 21C, the following apply: *p<0.05 vs. Control, #p<0.05 vs. ACM, $p<0.05 vs. ACM+BMP-7, n=5-7.

Atherosclerosis, an inflammatory disease leading to arterial wall thickening consequent to plaque formation and accumulation, is the leading cause of myocardial infarction and stroke (Libby et al., 2009). Monocytes play an elemental role in the development and progression of inflammatory diseases in that they migrate to damaged areas, divide and differentiate into macrophages. The ratio of M1 to M2 macrophages following monocyte polarization can determine the severity and progression of inflammatory disorders (Moore et al., 2011; Seimon et al., 2009; Galkina et al., 2009). To this end, M1 macrophage polarization was investigated as well as concentrations of pro-inflammatory cytokines Treatment with ACM and ACM and follistatin induced monocyte polarization towards the M1 phenotype, whereas treatment with BMP-7 dramatically decreased M1 macrophage yields, which indicated the potential of BMP-7 to prevent monocyte to M1 macrophage polarization. Congruent with increased M1 macrophage concentrations, pro-inflammatory cytokines (IL-6, TNF-α, and MCP-1) were significantly increased in the ACM and follistatin groups. Following BMP-7 administration, concentrations of IL-6, TNF-α and MCP-1 were significantly reduced. These data indicate a correlation between BMP-7, diminished M1 macrophage polarization, and reduced pro-inflammatory cytokine expression.

Quantification of anti-inflammatory M2 macrophage polarization revealed a significantly higher yield of M2 macrophages following BMP-7 treatment. Furthermore, in agreement with M2 macrophage yields, anti-inflammatory markers (arginase-1 and IL-10) were significantly increased following treatment with BMP-7. In the presence of BMP-7, monocytes were more readily polarizing into M2 macrophages and expressing anti-inflammatory cytokines but inhibition of BMP-7 by its inhibitor, follistatin, prevented that polarization and enhanced expression of anti-inflammatory cytokines. These data indicate that under pathological stressed conditions, monocytes, which exhibit high plasticity, undergo polarization into macrophages and treatment with certain factors can direct polarization and increase the ratio of M2 macrophages over M1 macrophages (Moore et al., 2011; Libby et al., 2009; Rocher et al., 2012; Wolfs et al., 2011; Lovren et al., 2010). By increasing polarization of M2 macrophages from monocytes and thereby increasing expression of anti-inflammatory cytokine markers, the beneficial potential of BMP-7 may include decreasing the progression of the inflammatory response and preventing further injury.

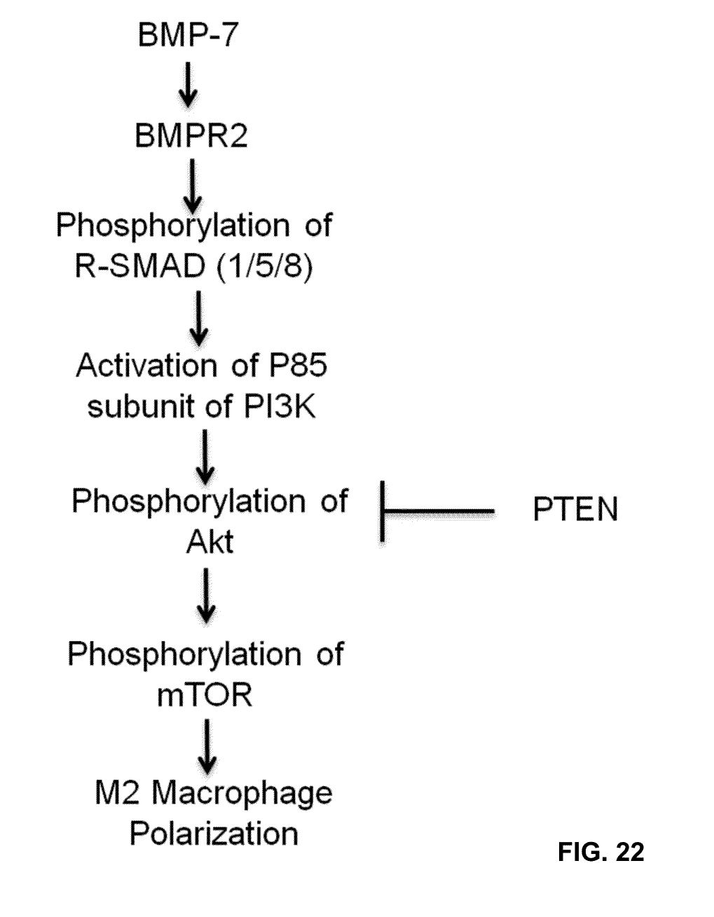

BMP-7 activates the SMAD-1, SMAD-5, and SMAD-8 pathway following binding to BMPR2 but studies have shown that it is also responsible for activation of other non-SMAD pathways such as the PI3K-Akt-mTOR pathway (Shimizu et al., 2012; Boon et al., 2011; Perron et al., 2009). The data presented herein show that BMP-7 upregulated and binded to BMPR2, phosphorylated SMAD-1, SMAD-5, and SMAD-8, and activated PI3K, which resulted in downstream activation of Akt and mTOR. (FIG. 22). The expression of p-PTEN, an inhibitor of the PI3K pathway, was significantly upregulated in the ACM and ACM+BMP-7+Foll groups but was significantly down-regulated following BMP-7 treatment. BMP-7 promoted PI3K signaling through upregulated mediators, and blocked inhibition of the signaling cascade (Weichhart et al., 2008). Furthermore, the inhibitor of BMP-7, follistatin, successfully prevented increased p-SMAD1/5/8 expression and significantly decreased expression of PI3K. These data indicate that the binding of BMP-7 to BMPR2 activated SMAD1/5/8 and subsequently PI3K.

Activation of the PI3K pathway controls production of transcriptional factors, which regulate key inflammatory cytokines resulting in (i) increased expression of anti-inflammatory markers such as arginase-1 and IL-10, and (ii) inhibition of the production of pro-inflammatory markers (Weichhart et al., 2008; Araki et al., 2011; Weichhart et al., 2009; Martin et al., 2003). Specifically, in bone marrow derived macrophages, activation of the PI3K pathway resulted in increased polarization of M2 macrophages (Rauh et al., 2005). The activation of the signaling pathway shown in the schematic of FIG. 22 following BMP-7 administration resulted in increased expression of anti-inflammatory cytokines and inhibited expression of pro-inflammatory cytokines, which promoted paracrine effects on monocytes and macrophages yielding increased M2 macrophage polarization.

BMP-7 treatment significantly increased expression of IL-10 and percentage of arginase-1 positive cells. Treatment with LY significantly decreased these levels, indicating that BMP-7 induced M2 macrophage polarization and that the release of anti-inflammatory cytokines were mediated by the PI3K pathway. The data presented herein demonstrated that BMP-7 induced M2 macrophage polarization.

### REFERENCES

The following references, to the extent that they provide exemplary procedural or other details supplementary to those set forth herein, are specifically incorporated herein by reference.
Amthor H, et al. (2002) Follistatin regulates bone morphogenetic protein-7 (BMP-7) activity to stimulate embryonic muscle growth. Dev Biol 243(1):115-127.
Araki K, et al. (2011) TOR in the immune system. Curr Opin Cell Biol 23(6):707-715.
Asakura E, et al. (1997) Intravenously administered macrophage colony-stimulating factor (M-CSF) specifically acts on the spleen, resulting in the increasing and activating spleen macrophages for cytokine production in mice. Immunopharmacology 37:7-14.
Babamusta F, et al. (2006) Angiotensin II infusion induces site-specific intra-laminar hemorrhage in macrophage colony-stimulating factor-deficient mice. Atherosclerosis 186:282-290.
Beltrami C A, et al. (1997) Proliferating cell nuclear antigen (PCNA), DNA synthesis and mitosis in myocytes following cardiac transplantation in man. J. Mol. Cell Cardiol. 29:2789-2802.
Boon M R, et al. (2011) Bone morphogenetic protein 7: a broad-spectrum growth factor with multiple target therapeutic potency. Cytokine Growth Factor Rev. 22:221-229.
Bradley E W, et al. (2008) Pathway crosstalk between Ras/Raf and PI3K in promotion of M-CSF-induced MEK/ERK-mediated osteoclast survival. J. Cell Biochem. 104:1439-1451.
Bragdon B, et al. (2011) Bone morphogenetic proteins: a critical review. Cell Signal. 23:609-620.
Daigneault M, et al. (2010) The identification of markers of macrophage differentiation in PMA-stimulated THP-1 cells and monocyte-derived macrophages. PLoS. One. 5:e8668.
Danesh S M, et al. (2009) BMP and BMP receptor expression during murine organogenesis. Gene Expr. Patterns. 9:255-265.
Dean C, et al. (2004) BMP-7 regulates branching morphogenesis of the lacrimal gland by promoting mesenchymal proliferation and condensation. Development 131:4155-4165.
Derynck R, et al. (2003) Smad-dependent and Smad-independent pathways in TGF-beta family signalling. Nature 425:577-584.
Desmyter S, et al. (2008) The role of bone morphogenetic protein-7 (Osteogenic Protein-1) in the treatment of tibial fracture non-unions. An overview of the use in Belgium. Acta Orthop. Belg. 74:534-537.
Devaraj S, et al. (2011) C-reactive protein polarizes human macrophages to an M1 phenotype and inhibits transformation to the M2 phenotype. Arterioscler. Thromb. Vasc. Biol. 31:1397-1402.
Galkina E, et al. (2009) Immune and inflammatory mechanisms of atherosclerosis (*). Annu Rev Immunol 27:165-197.
Glass C, et al. (2011) ES cells overexpressing microRNA-1 attenuate apoptosis in the injured myocardium. Mol. Cell Biochem. 357:135-141.

Glass C, et al. (2011) MicroRNA-1 transfected embryonic stem cells enhance cardiac myocyte differentiation and inhibit apoptosis by modulating the PTEN/Akt pathway in the infarcted heart. Am. J. Physiol Heart Circ. Physiol 301:H2038-H2049.
Gould S E, et al. (2002) BMP-7 regulates chemokine, cytokine, and hemodynamic gene expression in proximal tubule cells. Kidney Int. 61:51-60.
Hasegawa M, et al. (2009) A novel inhibitor of Smad-dependent transcriptional activation suppresses tissue fibrosis in mouse models of systemic sclerosis. Arthritis Rheum. 60:3465-3475.
Hosoda T, et al. (2009) Clonality of mouse and human cardiomyogenesis in vivo. Proc. Natl. Acad. Sci. U.S. A 106:17169-17174.
Houthuys E, et al. (2010) A method for the isolation and purification of mouse peripheral blood monocytes. J. Immunol. Methods 359:1-10.
Huse K, et al. (2011) Bone morphogenetic proteins inhibit CD40L/IL-21-induced Ig production in human B cells: differential effects of BMP-6 and BMP-7. Eur. J. Immunol. 41:3135-3145.
Iemura S, et al. (1998) Direct binding of follistatin to a complex of bone-morphogenetic protein and its receptor inhibits ventral and epidermal cell fates in early Xenopus embryo. Proc. Natl. Acad. Sci. USA 95:9337-9342.
Kadl A, et al. (2010) Identification of a novel macrophage phenotype that develops in response to atherogenic phospholipids via Nrf2. Circ. Res. 107:737-746.
Kigerl K A, et al. (2009) Identification of two distinct macrophage subsets with divergent effects causing either neurotoxicity or regeneration in the injured mouse spinal cord. J. Neurosci. 29:13435-13444.
Kleemann R, et al. (2008) Cytokines and atherosclerosis: a comprehensive review of studies in mice. Cardiovasc. Res. 79:360-376.
Kutza J, et al. (2000) Macrophage colony-stimulating factor antagonists inhibit replication of HIV-1 in human macrophages. J. Immunol. 164:4955-4960.
Libby P, et al. (2009) Inflammation in atherosclerosis: from pathophysiology to practice. Am Coll Cardiol 54(23):2129-2138.
Lovren F, et al. (2010) Adiponectin primes human monocytes into alternative anti-inflammatory M2 macrophages. Am J Physiol Heart Circ Physiol 299(3):H656-H663.
Maiseyeu A, et al. (2010) Detection of macrophages via paramagnetic vesicles incorporating oxidatively tailored cholesterol ester: an approach for atherosclerosis imaging. Nanomedicine. (Lond) 5:1341-1356.
Manson S R, et al. (2011) The BMP-7-Smad1/5/8 pathway promotes kidney repair after obstruction induced renal injury. J Urol 185(6 Suppl):2523-2530.
Maric I, et al. (2012) BMP signaling in rats with TNBS-induced colitis following BMP-7 therapy. Am J Physiol Gastrointest Liver Physiol 302(10):G1151-G1162.
Martin M, et al. (2003) Role of the phosphatidylinositol 3 kinase-Akt pathway in the regulation of IL-10 and IL-12 by Porphyromonas gingivalis lipopolysaccharide. J Immunol 171(2):717-725.
Martinez F O, et al. (2006) Transcriptional profiling of the human monocyte-to-macrophage differentiation and polarization: new molecules and patterns of gene expression. J. Immunol. 177:7303-7311.
Monzen K, et al. (1999) Bone morphogenetic proteins induce cardiomyocyte differentiation through the mitogen-activated protein kinase kinase kinase TAK1 and cardiac transcription factors Csx/Nkx-2.5 and GATA-4. Mol Cell Biol 19(10):7096-7105.
Moore K J, et al. (2011) Macrophages in the pathogenesis of atherosclerosis. Cell 145(3):341-355.
Moustakas A, et al. (2005) Non-Smad TGF-beta signals. J. Cell Sci. 118:3573-3584.
Nam D, et al. (2010) A model of disturbed flow-induced atherosclerosis in mouse carotid artery by partial ligation and a simple method of RNA isolation from carotid endothelium. J. Vis. Exp.
Nam D, et al. (2009) Partial carotid ligation is a model of acutely induced disturbed flow, leading to rapid endothelial dysfunction and atherosclerosis. Am. J. Physiol Heart Circ. Physiol 297:H1535-H1543.
Parthasarathy S, et al. (1988) Is oxidized low density lipoprotein involved in the recruitment and retention of monocyte/macrophages in the artery wall during the initiation of atherosclerosis? Basic Life Sci. 49:375-380.
Parthasarathy S, et al. (1994) Mechanisms of oxidation, antioxidants, and atherosclerosis. Curr. Opin. Lipidol. 5:371-375.
Perron J C, et al. (2009) ActRIIA and BMPRII Type II BMP receptor subunits selectively required for Smad4-independent BMP-7-evoked chemotaxis. PLoS One 4(12):e8198.
Qiao J H, et al. (1997) Role of macrophage colony-stimulating factor in atherosclerosis: studies of osteopetrotic mice. Am. J. Pathol. 150:1687-1699.
Rauh M J, et al. (2005) SHIP represses the generation of alternatively activated macrophages. Immunity 23(4):361-374.
Rocher C, et al. (2012) Bone morphogenetic protein 7 polarizes THP-1 cells into M2 macrophages. Can J Physiol Pharmacol 90(7):947-951.
Schulz T J, et al. (2011) Identification of inducible brown adipocyte progenitors residing in skeletal muscle and white fat. Proc. Natl. Acad. Sci. U.S. A 108:143-148.
Seimon T, et al. (2009) Mechanisms and consequences of macrophage apoptosis in atherosclerosis. J Lipid Res 50 Suppl:S382-5387.
Sekiya I, et al. (2009) Periodic knee injections of BMP-7 delay cartilage degeneration induced by excessive running in rats. J. Orthop. Res. 27:1088-1092.
Shimizu T, et al. (2012) Bone morphogenetic protein (BMP)-4 and BMP-7 suppress granulosa cell apoptosis via different pathways: BMP-4 via PI3K/PDK-1/Akt and BMP-7 via PI3K/PDK-1/PKC. Biochem Biophys Res Commun 417(2):869-873.
Sierra-Filardi E, et al. (2010) Heme Oxygenase-1 expression in M-CSF-polarized M2 macrophages contributes to LPS-induced IL-10 release. Immunobiology 215:788-795.
Singla D K, et al. (2007) Factors released from embryonic stem cells inhibit apoptosis of H9c2 cells. Am J Physiol Heart Circ Physiol 293(3):H1590-H1595.
Singla D K, et al. (2008) Factors released from embryonic stem cells inhibit apoptosis in H9c2 cells through PI3K/Akt but not ERK pathway. Am J Physiol Heart Circ Physiol 295(2):H907-H913.
Singla D K, et al. (2011) iPS Cells Repair and Regenerate Infarcted Myocardium. Mol. Pharm.
Singla D K, et al. (2011) TGF-beta2 treatment enhances cytoprotective factors released from embryonic stem cells and inhibits apoptosis in infarcted myocardium. Am. J. Physiol Heart Circ. Physiol 300:H1442-H1450.
Singla D K, et al. (2007) Transplanted embryonic stem cells following mouse myocardial infarction inhibit apoptosis and cardiac remodeling. Am. J. Physiol Heart Circ. Physiol 293:H1308-H1314.
Sinha P, et al. (2005) Interleukin-13-regulated M2 macrophages in combination with myeloid suppressor cells block immune surveillance against metastasis. Cancer Res 65(24):11743-11751.
Smith J D, et al. (1995) Decreased atherosclerosis in mice deficient in both macrophage colony-stimulating factor (op) and apolipoprotein E. Proc. Natl. Acad. Sci. U.S. A 92:8264-8268.
Sovershaev M A, et al. (2011) Increased expression of TF in BMP-7-treated human mononuclear cells depends on activation of select MAPK signaling pathways. Thromb. Res. 128:e154-e159.
Stebbins J L, et al. (2008) Identification of a new JNK inhibitor targeting the JNK-JIP interaction site. Proc. Natl. Acad. Sci. U.S. A 105:16809-16813.
Su J, et al. (2010) SB203580, a p38 inhibitor, improved cardiac function but worsened lung injury and survival during Escherichia coli pneumonia in mice. J. Trauma 68:1317-1327.
Tamaki Y, et al. (2008) Enhanced osteolytic potential of monocytes/macrophages derived from bone marrow after particle stimulation. J. Biomed. Mater. Res. B Appl. Biomater. 84:191-204.
Tardif G, et al. (2009) The BMP antagonists follistatin and gremlin in normal and early osteoarthritic cartilage: an immunohistochemical study. Osteoarthritis Cartilage 17(2):263-270.
Tseng Y H, et al. (2008) New role of bone morphogenetic protein 7 in brown adipogenesis and energy expenditure. Nature 454:1000-1004.
Wang S, et al. (2003) Bone morphogenic protein-7 (BMP-7), a novel therapy for diabetic nephropathy. Kidney Int 63(6):2037-2049.
Weichhart T, et al. (2008) The PI3K/Akt/mTOR pathway in innate immune cells: emerging therapeutic applications. Ann Rheum Dis 67 Suppl 3:iii70-iii74.
Weichhart T, et al. (2009) The multiple facets of mTOR in immunity. Trends Immunol 30(5):218-226.
Wolfs I M, et al. (2011) Differentiation factors and cytokines in the atherosclerotic plaque micro-environment as a trigger for macrophage polarisation. Thromb Haemost 106(5):763-771.
Woollard K J, et al. (2010) Monocytes in atherosclerosis: subsets and functions. Nat Rev Cardiol 7(2):77-86.
Xu W, et al. (2006) IL-10-producing macrophages preferentially clear early apoptotic cells. Blood 107(12):4930-4937.
Yakeu G, et al. (2010) Low-intensity exercise enhances expression of markers of alternative activation in circulating leukocytes: roles of PPARgamma and Th2 cytokines Atherosclerosis 212:668-673.
Yamamoto S, et al. (2011) Macrophage polarization by angiotensin II-type 1 receptor aggravates renal injury-acceleration of atherosclerosis. Arterioscler. Thromb. Vasc. Biol. 31:2856-2864.
Zeisberg M, et al. (2003) Bone morphogenic protein-7 inhibits progression of chronic renal fibrosis associated with two genetic mouse models. Am. J. Physiol Renal Physiol 285:F1060-F1067.

What is claimed is:

1. A method of modulating polarization of a population of monocytes in a subject, comprising: administering a composition comprising an effective amount of BMP-7 to a population of monocytes in a subject, thereby modulating the polarization of the monocytes.

2. The method of claim 1, wherein modulating the polarization of monocytes comprises increasing the rate of or the extent of the polarization of the monocytes into M2 macrophages.

3. The method of claim 1, wherein modulating the polarization of monocytes comprises decreasing the rate of or the extent of the polarization of monocytes into M1 macrophages.

4. The method of claim 1, wherein the subject is diagnosed with atherosclerosis.

5. The method of claim 1, further comprising administering to the subject at least one second agent that modulates the polarization of monocytes.

6. The method of claim 5, wherein the at least one second agent is M-CSF.

7. The method of claim 5, wherein the at least one second agent is not M-CSF.

8. The method of claim 7, further comprising administering to the subject M-CSF.

9. A method of ameliorating disease progression in a subject diagnosed with atherosclerosis, comprising: administering to a subject a composition comprising an effective amount of BMP-7; thereby modulating the polarization of infiltrating monocytes in a subject.

10. The method of claim 9, wherein modulating the polarization of monocytes comprises increasing the rate of or the extent of the polarization of the monocytes into M2 macrophages.

11. The method of claim 9, wherein modulating the polarization of monocytes comprises decreasing the rate of or the extent of the polarization of monocytes into M1 macrophages.

12. The method of claim 9, further comprising administering to a subject at least one second agent that modulates the polarization of monocytes.

13. The method of claim 12, wherein the at least one second agent is M-CSF.

14. The method of claim 12, wherein the at least one second agent is not M-CSF.

15. The method of claim 14, further comprising administering to the subject M-CSF.

16. The method of claim 9, wherein, following the administration of a composition comprising an effective amount of BMP-7, the severity of atherosclerosis is reduced, the blood velocity is improved, the formation of plaque is reduced, or a combination thereof.

17. The method of claim 9, wherein, following the administration of a comprising an effective amount of BMP-7, cardiac function is enhanced.

18. The method of claim 17, wherein enhanced cardiac function comprises (i) improving left ventricular function, (ii) improving fractional shortening, (iii) improving ejection fraction, (iv) reducing end-diastolic volume, (v) decreasing left ventricular mass, and (v) normalizing of heart geometry, or (vi) a combination thereof.

19. A composition, comprising: an effective amount of BMP-7, and a second agent that modulates the polarization of monocytes, M-CSF, or a combination thereof.

20. The composition of claim 19, further comprising a pharmaceutically acceptable carrier.
